(19) United States
(12) Patent Application Publication
(10) Pub. No.: US 2022/0168413 A1
(43) Pub. Date: Jun. 2, 2022

(54) ADJUVANTED MULTIVALENT INFLUENZA VACCINES

(71) Applicant: Seqirus UK Limited, Berkshire (GB)

(72) Inventors: Max CIARLET, Arlington, MA (US); Christian MANDL, Lexington, MA (US); Andrea FELLER, Cambridge, MA (US); Brett LEAV, Cambridge, MA (US); Gillis OTTEN, Cambridge, MA (US)

(21) Appl. No.: 17/432,986

(22) PCT Filed: Feb. 24, 2020
(86) PCT No.: PCT/IB2020/000207

§ 371 Date: Aug. 23, 2021

Related U.S. Application Data
(60) Provisional application No. 62/809,914, filed on Feb. 25, 2019.

(51) Int. Cl. A61K 39/145; A61P 31/16; C12N 7/00

(52) U.S. Cl. CPC A61K 39/145; A61K 2039/5252; C12N 7/00; A61P 31/16

(57) ABSTRACT

The present disclosure relates to vaccine compositions comprising a) antigens from at least three different strains of influenza vims, preferably at least four different strains of influenza vims, and b) an oil-in-water emulsion adjuvant, wherein the amount of the oil-in-water emulsion adjuvant is greater than an amount of an oil-in-water emulsion adjuvant in a standard-dose adjuvanted multivalent influenza vaccine. Additionally, the total amount of the antigens in the vaccine compositions may be greater than a total amount of antigens in a standard-dose adjuvanted multivalent influenza vaccine. In preferred aspects, the present disclosure further describes uses of these vaccine compositions for safe and effective induction of immune responses in adults at least 65 years of age.

### FIELD

The present disclosure relates to vaccine compositions comprising a) antigens from at least three different strains of influenza virus, preferably at least four different strains of influenza virus, and b) an oil-in-water emulsion adjuvant, wherein the amount of the oil-in-water emulsion adjuvant is greater than an amount of an oil-in-water emulsion adjuvant in a standard-dose adjuvanted multivalent influenza vaccine. Additionally, the total amount of the antigens in the vaccine compositions may be greater than a total amount of antigens in a standard-dose adjuvanted multivalent influenza vaccine. In preferred aspects, the present disclosure further describes uses of these vaccine compositions for safe and effective induction of immune responses in adults at least 65 years of age.

### BACKGROUND

During the 2017-2018 season, rates of influenza-related hospitalizations and deaths increased dramatically among the elderly [1]. Even in years without mismatch, influenza-related hospitalizations for pneumonia and cardiac disease are significantly higher among adults 65 years of age, and 90% of seasonal influenza-related deaths occur in this population [2-4]. Age-related immune dysfunction in older adults is believed to contribute to their vulnerability to influenza infection and may also compromise the effectiveness of conventional inactivated influenza vaccines [4-6].

Oil-in-water emulsions have been found to be suitable for use in adjuvanting influenza virus vaccines. For example, the addition of the squalene-based oil-in-water adjuvant MF59 to the influenza vaccine has been shown to increase vaccine immunogenicity [6-8]. MF59 increases antigen uptake, macrophage recruitment, and lymph node migration and broadens the spectrum of antibody recognition of hemagglutinin epitopes [9-14]. An MF59-adjuvanted trivalent inactivated influenza vaccine (aTIV; Fluad™ [Seqirus Vaccines Limited f/k/a Novartis Vaccines]) has been licensed in Europe since 1997 and in the United States since 2015. In studies with older adults, vaccination with aTIV reduced the risk of laboratory-confirmed influenza as well as hospitalization for influenza or pneumonia when compared with conventional trivalent inactivated influenza vaccine (TIV) [15, 16].

The immunogenicity of aTIV in adults 65 years of age has been evaluated since the 1990s. A study involved >7000 elderly persons and demonstrated higher immune responses compared with conventional non-adjuvanted TIV [7]. However, whether increased dosages of an oil-in-water adjuvant (e.g., MF59) would enhance the immune response in adults 65 years of age is unknown. In addition, it remains unknown whether increased dosages of an oil-in-water adjuvant can be combined with increased dosages of the influenza antigens to enhance immunogenicity in older adults without causing major adverse effects.

Accordingly, there remains a need for new vaccine compositions for safe and effective induction of immune responses in adults 65 years of age.

### SUMMARY

The disclosure provides for vaccine compositions comprising antigens from at least three different strains of influenza virus, preferably at least four different strains of influenza virus, and an oil-in-water emulsion adjuvant such as MF59, wherein the amount of the oil-in-water emulsion adjuvant is greater than an amount of an oil-in-water emulsion adjuvant in a standard-dose adjuvanted multivalent influenza vaccine. Additionally, the total amount of the antigens in the vaccine compositions may be greater than a total amount of antigens in a standard-dose adjuvanted multivalent influenza vaccine.

The disclosure provides for methods for safe and effective induction of an immune response in a human at least 65 years of age, comprising administering a vaccine composition according to this disclosure to the human. The disclosure also provides for methods for enhancing immunogenicity in a human at least 65 years of age, comprising administering a vaccine composition according to this disclosure to the human, preferably the administration of the vaccine composition does not increase the incidence of unsolicited adverse events or systemic solicited adverse events.

The disclosure also provides for methods for producing a vaccine composition, comprising admixing antigens from at least three different strains of influenza virus, preferably at least four different strains of influenza virus, and an oil-in-water emulsion adjuvant, wherein the amount of the oil-in-water emulsion adjuvant is greater than an amount of an oil-in-water emulsion adjuvant in a standard-dose adjuvanted multivalent influenza vaccine. Additionally, the total amount of the antigens in the vaccine compositions may be greater than a total amount of antigens in a standard-dose adjuvanted multivalent influenza vaccine.

### DESCRIPTION OF DRAWINGS

FIG. 1 depicts information on patient disposition.

FIG. 2 depicts hemagglutination inhibition (HI) geometric mean titres (GMTs) for A/H3N2 by study visit. Groups 1˜4 received a single intramuscular vaccine injection containing the amounts of MF59 and haemagglutinin (HA) antigen listed for each group in the non-dominant arm. Groups 5 and 6 received vaccine containing the amounts of MF59 and HA listed in the left deltoid and saline in the right deltoid. Group 7 received one vaccine injection containing 9.75 mg of MF59 and 45 μg HA in each arm, for a total of 19.5 mg of MF59 and 90 μg HA. T-bars represent 95% confidence intervals.

FIG. 3 depicts hemagglutination inhibition (HI) geometric mean ratios (GMR; Day 22/Day 1) for A/H1N1, A/H3N2, and B strain plotted by dosage of MF59 and haemagglutinin (HA) antigen content. Data shown are from Groups 1-4 and 6. T-bars represent 95% confidence intervals.

FIG. 4 depicts percentage of subjects with seroconversion (pre-vaccination hemagglutination inhibition [HI] titre <10 and a post-vaccination HI titre ≥40) or significant increase (pre-vaccination HI titre 0 and a minimum 4-fold rise in post-vaccination HI antibody titre) in HI titre at Day 22. T-bars represent 95% confidence intervals.

FIG. 5 depicts microneutralization (MN) assay geometric mean ratios (GMR; Day 22/Day 1) for A/H1N1, A/H3N2, and B strain plotted by dosage of MF59 and haemagglutinin (HA) antigen content. Data shown are from Groups 1-4 and 6. T-bars represent 95% confidence intervals.

FIG. 6 depicts percentage of subjects with 4-fold increase in microneutralization (MN) titre at Day 22.

FIG. 7 depicts representative quadrivalent vaccine compositions according to this disclosure (vaccine #2 to vaccine #12) compared to a standard-dose adjuvanted quadrivalent influenza vaccine (aQIVc; vaccine #1).

### DETAILED DESCRIPTION

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in the same manner as the term “comprising.”

As used herein, the term “administering” refers to the placement of a vaccine composition into a subject by a method or route that results in at least partial localization of the vaccine composition at a desired site or tissue location. For example, a vaccine composition may be administered to the subject by intramuscular injection, subcutaneous delivery, intranasal delivery, oral delivery, intradermal delivery, transdermal delivery, transcutaneous delivery, or topical route.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition that “comprises,” “has” or “includes” one or more features is not limited to possessing only those one or more features and can cover other unlisted features. The use of any and all examples, or exemplary language (e.g., “such as”) provided with respect to certain embodiments herein is intended merely to better illuminate the present disclosure and does not pose a limitation on the scope of the present disclosure otherwise claimed.

1. Influenza vaccine compositions comprising a) antigens from at least three different strains of influenza virus, preferably at least four different strains of influenza virus, and b) an oil-in-water emulsion adjuvant

According to the present disclosure, a vaccine composition comprises antigens from at least three different strains of influenza virus, preferably at least four different strains of influenza virus, and an oil-in-water emulsion adjuvant, wherein the amount of the oil-in-water emulsion adjuvant is greater than an amount of an oil-in-water emulsion adjuvant in a standard-dose adjuvanted multivalent influenza vaccine. Furthermore, in some embodiments, the total amount of the antigens in the vaccine compositions may be greater than a total amount of antigens in a standard-dose adjuvanted multivalent influenza vaccine.

Antigen

Vaccine compositions according to this disclosure include antigens from suitable influenza virus strains. In some embodiments, the antigen may be included in a sub-virion form, e.g., in the form of a split virus, where the viral lipid envelope has been dissolved or disrupted, or in the form of one or more purified viral surface proteins (subunits). In some embodiments, the antigen may be from a whole virus, e.g., a live attenuated whole virus, or an inactivated whole virus. Further details on influenza vaccine antigens can be found in chapters 17 & 18 of Vaccines (eds. Plotkin & Orenstein, 4th edition, 2004, ISBN 0-7216-9688-0).

Methods of splitting influenza viruses are well known in the art. See, e.g., WO 02/28422, WO 02/067983, WO 02/074336, and WO 01/21151. Splitting of the virus is carried out by disrupting or fragmenting whole virus, whether infectious (wild-type or attenuated) or non-infectious (e.g., inactivated), with a disrupting concentration of a splitting agent. The disruption results in a full or partial solubilization of the virus proteins, altering the integrity of the virus. Splitting agents can be non-ionic or ionic (e.g., cationic) surfactants, e.g., alkylglycosides, alkylthioglycosides, acyl sugars, sulphobetaines, betains, polyoxyethylenealkylethers, N,N-dialkyl-Glucamides, Hecameg, alkylphenoxy-polyethoxyethanols, quaternary ammonium compounds, sarcosyl, CTABs (cetyl trimethyl ammonium bromides), tri-N-butyl phosphate, Cetavlon, myristyltrimethylammonium salts, lipofectin, lipofectamine, and DOT-MA, the octyl- or nonylphenoxy polyoxyethanols (e.g., the Triton surfactants, such as Triton X-100), polyoxyethylene sorbitan esters (the Tween surfactants), polyoxyethylene ethers, or polyoxyethlene esters.

Methods of purifying viral surface proteins (subunits) from influenza viruses are well known. Vaccines based on purified viral proteins typically include the hemagglutinin (HA) protein, and often include the neuraminidase (NA) protein as well. Processes for preparing these proteins in purified form are well known in the art. In some embodiments, a vaccine composition according to this disclosure comprises HA antigens (see Example 1). HA may be a natural HA as found in a virus, or may have been modified. For instance, it is known to modify HA to remove determinants (e.g., hyper-basic regions around the cleavage site between HA1 and HA2) that cause a virus to be highly pathogenic in avian species, as these determinants can otherwise prevent a virus from being grown in eggs.

As a further alternative, the vaccine may include antigens from a whole virus, e.g., a live attenuated whole virus, or an inactivated whole virus. Methods of inactivating or killing viruses to destroy their ability to infect mammalian cells are known in the art. Such methods include both chemical and physical means. Chemical means for inactivating a virus include treatment with an effective amount of one or more of the following agents: detergents, formaldehyde, formalin, β-propiolactone, or UV light. Additional chemical means for inactivation include treatment with methylene blue, psoralen, carboxyfullerene (C60), or a combination of any thereof. Other means of viral inactivation, including binary ethylamine, acetyl ethyleneimine, and gamma irradiation, are well known in the art.

The use of recombinant DNA technology to produce subunit vaccines involves molecular cloning and expression in an appropriate vector of generic information coding for the antigens which can elicit an immune response. Techniques of molecular cloning and expression are well-known in the art. Thus, in some embodiments, vaccine compositions according to this disclosure include recombinant antigens.

The antigens may be from any suitable influenza virus, including influenza A, B and C viruses. It is particularly useful to use strains of influenza A virus that can infect humans. Influenza virus strains for use in vaccines change from season to season. Thus, depending on the season, the vaccine may include influenza A virus HA subtypes H1, H2, H3, H4, H5, H6, H7, H8, H9, H10, H11, H12, H13, H14, H15, or H16. In addition, the vaccine may have any of influenza NA subtypes N1, N2, N3, N4, N5, N6, N7, N8, or N9. In some embodiments, the vaccine comprises trivalent antigens as follows: from an H1 virus strain, from an H3 virus strain, and from a B virus strain. In some embodiments, the vaccine comprises trivalent antigens as follows: from an H1N1 strain, from an H3N2 strain, and from a B virus strain such as B/Yamagata or B/Victoria strain. In some embodiments, the vaccine comprises quadrivalent antigens as follows: from an H1 virus strain, from an H3 virus strain, from a B virus strain, and from a different B virus strain. In some embodiments, the vaccine comprises quadrivalent antigens as follows: from an H1N1 virus strain, from an H3N2 virus strain, from a B/Yamagata strain, and from a B/Victoria strain.

Vaccine compositions according to this disclosure comprise antigens from at least three different strains of influenza virus, preferably at least four different strains of influenza virus, including those from influenza A virus and/or influenza B virus. In some embodiments, the vaccine composition comprises antigens from two influenza A virus strains and one influenza B virus strain. In some embodiments, the vaccine composition comprises antigens from two influenza A virus strains and two influenza B virus strains.

For example, in some embodiments, vaccine compositions according to this disclosure comprise antigens from two influenza A strains (H1N1 and H3N2) and one influenza B strain. In preferred embodiments, vaccine compositions according to this disclosure comprise antigens from at least four different influenza virus strains. The different strains will typically be grown separately and then mixed after the viruses have been harvested and antigens have been prepared. Thus a process of the invention may include the step of mixing antigens from more than one influenza strain.

In some embodiments, the four strains will include two influenza A virus strains and two influenza B virus strains (“A-A-B-B”). In other embodiments, the four strains will include three influenza A virus strains and one influenza B virus strain (“A-A-A-B”).

In some embodiments, a quadrivalent A-A-A-B vaccine may include antigens from an H1N1 strain, an H3N2 strain, an H5 strain (e.g., an H5N1 strain), and an influenza B strain. In some embodiments, a quadrivalent A-A-B-B vaccine may include antigens (from: (i) an H1N1 strain; (ii) an H3N2 strain; (iii) a B/Victoria strain; and (iv) a B/Yamagata strain.

Some recent influenza strains used in the manufacture of influenza vaccines are listed below:
an A/Michigan/45/2015 (H1N1) pdm09-like virus strain
an A/Singapore/INFIMH-16-0019/2016 (H3N2)-like virus strain
a B/Colorado/06/2017-like virus (B/Victoria lineage) strain
a B/Phuket/3073/2013-like virus (B/Yamagata lineage) strain.

Additional information on suitable influenza strains can be found at FDA's webpage, for example, www.fda.gov/BiologicsBloodVaccines/GuidanceComplianceRegulatoryInformation/Post-MarketActivities/LotReleases/ucm613863.htm.

The viruses used as the source of the antigens can be grown either on eggs or on cell culture. For example, one method for influenza virus growth uses specific pathogen-free (SPF) embryonated hen eggs, with virus being purified from the egg contents (allantoic fluid). More recently, however, viruses have been grown in animal cell culture and, for reasons of speed and patient allergies, this growth method is preferred. If egg-based viral growth is used, then one or more amino acids may be introduced into the allantoid fluid of the egg together with the virus.

When cell culture is used, the viral growth substrate will typically be a cell line of mammalian origin. Suitable mammalian cells of origin include, but are not limited to, hamster, cattle, primate (including humans and monkeys) and dog cells. Various cell types may be used, such as kidney cells, fibroblasts, retinal cells, and lung cells. For example, MDCK cell lines are suitable for growing influenza viruses. The original MDCK cell line is available from the ATCC as CCL 34, but derivatives of this cell line may also be used.

For growth on a cell line, such as on MDCK cells, virus may be grown on cells in suspension or in adherent culture. One suitable MDCK cell line for suspension culture is MDCK 33016 (deposited as DSM ACC 2219). As an alternative, microcarrier culture can be used. Cell lines supporting influenza virus replication may be grown in serum free culture media and/or protein free media. A medium is referred to as a serum-free medium in the context of the present invention in which there are no additives from serum of human or animal origin. Protein-free is understood to mean cultures in which multiplication of the cells occurs with exclusion of proteins, growth factors, other protein additives and non-serum proteins, but can optionally include proteins such as trypsin or other proteases that may be necessary for viral growth.

Additional examples of suitable cell lines as well as cell culturing methods can be found in WO 2007/052055, which is incorporated herein by reference in its entirety.

Where virus has been grown on a mammalian cell line, then the vaccine composition will advantageously be free from egg proteins (e.g., ovalbumin and ovomucoid) and from chicken DNA, thereby reducing allergenicity. The avoidance of allergens is a further way of minimizing Th2 responses.

Where virus has been grown on a cell line, then the vaccine composition may contain less than 10 ng (preferably less than 1 ng, and more preferably less than 100 pg) of residual host cell DNA per dose, although trace amounts of host cell DNA may be present. In general, the host cell DNA that it is desirable to exclude from compositions of the invention is DNA that is longer than 100 bp. Methods for measuring residual host cell DNA are well known in the art.

Where virus has been grown on a cell line, then the vaccine composition may contain hemagglutinin with a binding preference for oligosaccharides with a Sia(α2,6)Gal terminal disaccharide compared to oligosaccharides with a Sia(α2,3)Gal terminal disaccharide. Human influenza viruses bind to receptor oligosaccharides having a Sia(α2,6)Gal terminal disaccharide (sialic acid linked α-2,6 to galactose), but eggs and Vero cells have receptor oligosaccharides with a Sia(α2,3)Gal terminal disaccharide. Growth of human influenza viruses in cells such as MDCK provides selection pressure on hemagglutinin to maintain the native Sia(α2,6)Gal binding, unlike egg passaging.

To determine if a virus has a binding preference for oligosaccharides with a Sia(α2,6)Gal terminal disaccharide compared to oligosaccharides with a Sia(α2,3)Gal terminal disaccharide, various assays known in the art can be used. See US 2011/0045022 A1, which is incorporated herein by reference in its entirety.

In some embodiments influenza strains used according to this disclosure have glycoproteins (including hemagglutinin) with a different glycosylation pattern from egg-derived viruses. Thus the glycoproteins will include glycoforms that are not seen in chicken eggs.

In all types of vaccine, dosage is typically normalized to 15 μg of HA per strain per dose. Normalization of doses is generally achieved by measuring concentrations using a single radial immunodiffusion (SRID) assay. Existing vaccines typically contain about 15 pg of HA per strain. See, e.g., aTIV used in Example 1.

Oil-In-Water Emulsion Adjuvant

Oil-in-water emulsions have been found to be suitable for use as adjuvants in influenza virus vaccines. Various such emulsions are known, and they typically include at least one oil and at least one surfactant, with the oil(s) and surfactant(s) being biodegradable (metabolizable) and biocompatible. The oil droplets in the emulsion are generally less than 5 μm in diameter, and may even have a sub-micron diameter, with these small sizes being achieved with a microfluidizer to provide stable emulsions. Droplets with a size less than 220 nm are preferred as they can be subjected to filter sterilization.

The oils can be from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used, e.g., obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene. Squalane, the saturated analog to squalene, is another example of oil. Fish oils, including squalene and squalane, are readily available from commercial sources or may be obtained by methods known in the art.

Surfactants can be classified by their hydrophile/lipophile balance (“HLB”). In some embodiments, the surfactants have a HLB of at least 10, preferably at least 15, and more preferably at least 16. Non-limiting examples of surfactants include: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin, and Triton X-100.

Mixtures of surfactants can be used, e.g., Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Suitable amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Whatever the choice of oil(s) and surfactant(s), the surfactant(s) is/are included in excess of the amount required for emulsification, such that free surfactant remains in the aqueous phase. Free surfactant in the final emulsion can be detected by various assays. For instance, a sucrose gradient centrifugation method can be used to separate emulsion droplets from the aqueous phase, and the aqueous phase can then be analyzed. Centrifugation can be used to separate the two phases, with the oil droplets coalescing and rising to the surface, after which the surfactant content of the aqueous phase can be determined, e.g., using HPLC or any other suitable analytical technique.

Specific oil-in-water emulsion adjuvants according to this disclosure include, but are not limited to, the following:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as “MF59” (see, e.g., Example 1). The MF59 emulsion advantageously includes citrate ions, e.g., 10 mM sodium citrate buffer. In some embodiments, the oil-in-water elusion adjuvant is a squalene-in-water emulsion adjuvant having 9.75 mg squalene.
An emulsion of squalene, a tocopherol, and Tween 80. The emulsion may include phosphate buffered saline. It may also include Span 85 (e.g., at 1%) and/or lecithin. These emulsions may have from 2 to 10% squalene, from 2 to 10% tocopherol and from 0.3 to 3% Tween 80, and the weight ratio of squalene:tocopherol is preferably ≤1 as this provides a more stable emulsion. Squalene and Tween 80 may be present volume ratio of about 5:2. One such emulsion can be made by dissolving Tween 80 in PBS to give a 2% solution, then mixing 90 mL of this solution with a mixture of (5 g of DL-α-tocopherol and 5 mL squalene), then microfluidizing the mixture. The resulting emulsion may have submicron oil droplets, e.g. with an average diameter of between 100 and 250 nm, preferably about 180 nm.
An emulsion of squalene, a tocopherol, and a Triton detergent (e.g., Triton X-100). The emulsion may also include a 3d-MPL. The emulsion may contain a phosphate buffer.
An emulsion comprising a polysorbate (e.g., polysorbate 80), a Triton detergent (e.g., Triton X-100) and a tocopherol (e.g., an α-tocopherol succinate). The emulsion may include these three components at a mass ratio of about 75:11:10 (e.g., 750 μg/mL polysorbate 80, 110 μg/mL Triton X-100 and 100 μg/mL α-tocopherol succinate), and these concentrations should include any contribution of these components from antigens. The emulsion may also include squalene. The emulsion may also include a 3d-MPL. The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant (0.05-1% Thr-MDP, 5% squalane, 2.5% Pluronic L121 and 0.2% polysorbate 80). It can also be used without the Thr-MDP, as in the “AF” adjuvant (5% squalane, 1.25% Pluronic L121 and 0.2% polysorbate 80).
An emulsion having from 0.5-50% of an oil, 0.1-10% of a phospholipid, and 0.05-5% of a non-ionic surfactant. Preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolizable oil (such as light mineral oil) and at least one surfactant (such as lecithin, Tween 80 or Span 80). Additives may be included, such as QuilA saponin, cholesterol, a saponin-lipophile conjugate (such as GPI-0100, produced by addition of aliphatic amine to desacylsaponin via the carboxyl group of glucuronic acid), dimethyidioctadecylammonium bromide and/or N,N-dioctadecyl-N,N-bis (2-hydroxyethyl)propanediamine.
An emulsion in which a saponin (e.g., QuilA or QS21) and a sterol (e.g., a cholesterol) are associated as helical micelles.

The emulsions and split antigen may be mixed during manufacture, before packaging, or they may be mixed extemporaneously, at the time of delivery. Thus, the adjuvant and antigen may be kept separately in a packaged or distributed vaccine, ready for final formulation at the time of use. The antigen will generally be in an aqueous form, such that the vaccine is finally prepared by mixing two liquids. The volume ratio of the two liquids for mixing can vary (e.g., between 5:1 and 1:5) but is generally about 1:1. After the antigen and adjuvant have been mixed, HA antigen will generally remain in aqueous solution but may distribute itself around the oil/water interface. In general, little if any HA antigen will enter the oil phase of the emulsion.

Where a vaccine composition includes a tocopherol, any of the α, β, γ, δ, ε or ξ tocopherols can be used, and α-tocopherols are preferred. The tocopherol can take several forms, e.g., different salts and/or isomers. Salts include organic salts, such as succinate, acetate, and nicotinate. D-α-tocopherol and DL-α-tocopherol can both be used. Tocopherols are advantageously included in vaccines for use in elderly adults (e.g., aged 65 years or older) because vitamin E has been reported to have a positive effect on the immune response in this patient group. They also have antioxidant properties that may help to stabilize the emulsions. A preferred α-tocopherol is DL-α-tocopherol, and the preferred salt of this tocopherol is the succinate. The succinate salt has been found to cooperate with TNF-related ligands in vivo. Moreover, α-tocopherol succinate is known to be compatible with influenza vaccines and to be a useful preservative as an alternative to mercurial compounds. In addition, vitamin E stimulation of immune cells can directly lead to increased IL-2 production (i.e., a Th1-type response), which may help to avoid an overt Th2 phenotype.

Additional Components

Vaccine compositions according to this disclosure may include components in addition to antigens and oil-in-water emulsion adjuvant. For example, they typically include one or more pharmaceutical carrier(s) and/or excipient(s) commonly used in the art.

The vaccine composition may include preservatives such as thiomersal or 2-phenoxyethanol. It is preferred, however, that the vaccine composition should be substantially free from (i.e., less than 5 μg/mL) mercurial material. Vaccines containing no mercury are more preferred, and this can conveniently be achieved when using a tocopherol-containing adjuvant by following the methods commonly used in the art. Preservative-free vaccines are preferred.

To control tonicity, the vaccine composition may include a physiological salt, such as a sodium salt. For example, sodium chloride (NaCl) may be present at between 1 and 20 mg/mL. Other salts that may be present include potassium chloride, potassium dihydrogen phosphate, disodium phosphate dehydrate, magnesium chloride, and calcium chloride.

Vaccine compositions may include one or more buffers. Typical buffers include: a phosphate buffer, a Tris buffer, a borate buffer, a succinate buffer, a histidine buffer, or a citrate buffer. The pH of a vaccine composition is generally between 5.0 and 8.1, and more typically between 6.0 and 8.0, e.g., 6.5 and 7.5, or between 7.0 and 7.8.

The vaccine composition is preferably sterile. The vaccine composition is preferably non-pyrogenic, e.g., containing <1 EU (endotoxin unit, a standard measure) per dose, and preferably <0.1 EU per dose. The vaccine composition is preferably gluten free.

Vaccine Compositions

A vaccine composition according to this disclosure comprises antigens from at least three different strains of influenza virus, preferably at least four different strains of influenza virus, and an oil-in-water emulsion adjuvant (e.g., MF59), wherein the amount of the oil-in-water emulsion adjuvant is greater than an amount of an oil-in-water emulsion adjuvant in a standard-dose adjuvanted multivalent influenza vaccine. Additionally, the total amount of the antigens in the vaccine compositions may be greater than a total amount of antigens in a standard-dose adjuvanted multivalent influenza vaccine.

Standard-dose multivalent influenza vaccines are well known in the art. In at least one aspect, standard-dose multivalent influenza vaccines are generally recognized by FDA (see, e.g., www.fda.gov/BiologicsBloodVaccines/GuidanceComplianceRegulatoryInformatio n/Post-MarketActivities/LotReleases/ucm062928.htm) and nominally contain 15 μg of HA per strain per dose. For example, the trivalent TIV used in Example 1 contains 15 μg of HA from each of A/California/7/2009 (H1N1) pdm09-like virus, A/Texas/50/2012 (H3N2)-like virus, and B/Massachusetts/2/2012-like virus.

Some known standard-dose multivalent influenza vaccines are listed below:
Fluzone (Trivalent or Quadrivalent)
Fluarix (Trivalent or Quadrivalent)
FluLaval (Trivalent or Quadrivalent).

Accordingly, in some embodiments, a vaccine composition according to this disclosure comprises antigens from at least three different strains of influenza virus, preferably at least four different strains of influenza virus, and an oil-in-water emulsion adjuvant, wherein the amount of the oil-in-water emulsion adjuvant is greater than an amount of an oil-in water emulsion adjuvant in a standard-dose adjuvanted multivalent influenza vaccine and no greater than three-fold of the amount of an oil-in water emulsion adjuvant in a standard-dose adjuvanted multivalent influenza vaccine. In some embodiments, the amount of the oil-in-water emulsion adjuvant is greater than the amount of an oil-in water emulsion adjuvant in a standard-dose adjuvanted multivalent influenza vaccine and no greater than two-fold of the amount of an oil-in water emulsion adjuvant in a standard-dose adjuvanted multivalent influenza vaccine. In some embodiments, the amount of the oil-in-water emulsion adjuvant is from two-fold to three-fold of the amount of an oil-in water emulsion adjuvant in a standard-dose adjuvanted multivalent influenza vaccine. In all of these embodiments, the total amount of the antigens is from one-fold to four-fold, from one-fold to three-fold, or from one-fold to two-fold, of the total amount of antigens in a standard-dose adjuvanted multivalent influenza vaccine. In all of these embodiments, the oil-in-water emulsion adjuvant may be a squalene-in-water emulsion adjuvant, e.g., MF59.

In some embodiments, a vaccine composition according to this disclosure comprises antigens from at least three different strains of influenza virus, wherein the standard-dose adjuvanted multivalent influenza vaccine is a trivalent influenza vaccine comprising antigens from two influenza A virus strains and one influenza B virus strain. In all of these embodiments, the total amount of the antigens is from one-fold to four-fold of a total amount of antigens in a standard-dose adjuvanted trivalent influenza vaccine, and the amount of the oil-in-water emulsion adjuvant is greater than an amount of an oil-in-water emulsion adjuvant in a standard-dose adjuvanted multivalent influenza vaccine and no greater than three-fold of the amount of an oil-in-water emulsion adjuvant in a standard-dose adjuvanted trivalent influenza vaccine. In some embodiments, the oil-in-water emulsion adjuvant may be a squalene-in-water emulsion adjuvant, e.g., MF59. In some embodiments, the standard-dose adjuvanted trivalent influenza vaccine comprises about 15 μg hemagglutinin (HA) from each of the three influenza virus strains. In some embodiments, the standard-dose adjuvanted trivalent influenza vaccine comprises from about 5 μg to about 30 μg, from about 5 μg to about 25 μg, from about 5 μg to about 20 μg, from about 5 μg to about 15 μg, from about 5 μg to about 10 μg, from about 10 μg to about 30 μg, from about 10 μg to about 25 μg, from about 10 μg to about 20 μg, from about 10 μg to about 15 μg, from about 15 μg to about 30 μg, from about 15 μg to about 25 μg, from about 15 μg to about 20 μg, from about 20 μg to about 30 μg, from about 20 μg to about 25 μg, or from about 25 μg to about 30 μg hemagglutinin (HA) from each of the three influenza virus strains.

In accordance with preferred embodiments, the standard-dose adjuvanted multivalent influenza vaccine comprises about 15 μg from hemagglutinin (HA) from each of the influenza virus strains. In additional preferred embodiment, the standard-dose adjuvanted multivalent influenza vaccine comprises at least about 15 μg from hemagglutinin (HA) from each of the influenza virus strains.

In some embodiments, a vaccine composition according to this disclosure comprises antigens from at least four different strains of influenza virus, wherein the standard-dose adjuvanted multivalent influenza vaccine is a quadrivalent influenza vaccine comprising antigens from two influenza A virus strains and two influenza B virus strains. In all of these embodiments, the total amount of antigens is from one-fold to four-fold of a total amount of antigens in a standard-dose adjuvanted quadrivalent influenza vaccine, and the amount of the oil-in-water emulsion adjuvant is greater than an amount of an oil-in-water emulsion adjuvant in a standard-dose adjuvanted multivalent influenza vaccine and no greater than three-fold of the amount in a standard-dose adjuvanted quadrivalent influenza vaccine. In some embodiments, the oil-in-water emulsion adjuvant may be a squalene-in-water emulsion adjuvant, e.g., MF59. In some embodiments, the standard-dose adjuvanted quadrivalent influenza vaccine comprises about 15 μg hemagglutinin (HA) from each of the four influenza virus strains. In some embodiments, the standard-dose adjuvanted multivalent influenza vaccine comprises from about 5 μg to about 30 μg, from about 5 μg to about 25 μg, from about 5 μg to about 20 μg, from about 5 μg to about 15 μg, from about 5 μg to about 10 μg, from about 10 μg to about 30 μg, from about 10 μg to about 25 μg, from about 10 μg to about 20 μg, from about 10 μg to about 15 μg, from about 15 μg to about 30 μg, from about 15 μg to about 25 μg, from about 15 μg to about 20 μg, from about 20 μg to about 30 μg, from about 20 μg to about 25 μg, or from about 25 μg to about 30 μg hemagglutinin (HA) from each of the four influenza virus strains.

In some exemplary embodiments, described in in Example 1, a trivalent vaccine composition according to this disclosure may comprise:
1-fold HA and 2-fold MF59
2-fold HA and 1-fold MF59
2-fold HA and 2-fold MF59
1-fold HA and 3-fold MF59.

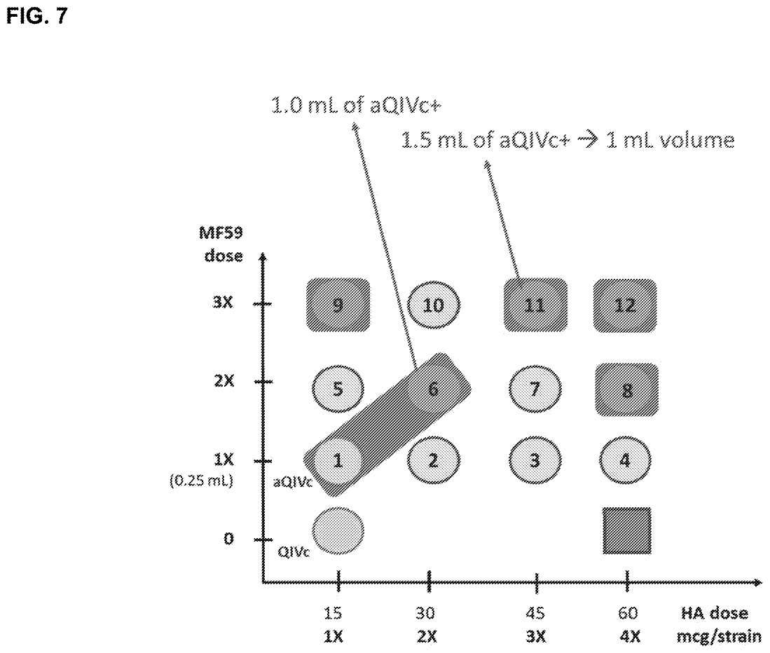

In additional exemplary preferred embodiments, described in Example 2 and shown in FIG. 7, a quadrivalent vaccine composition according to this disclosure may comprise:
2-fold HA and 1-fold MF59 (vaccine #2)
3-fold HA and 1-fold MF59 (vaccine #3)
4-fold HA and 1-fold MF59 (vaccine #4)
1-fold HA and 2-fold MF59 (vaccine #5)
2-fold HA and 2-fold MF59 (vaccine #6)
3-fold HA and 2-fold MF59 (vaccine #7)
4-fold HA and 2-fold MF59 (vaccine #8)
1-fold HA and 3-fold MF59 (vaccine #9)
2-fold HA and 3-fold MF59 (vaccine #10)
3-fold HA and 3-fold MF59 (vaccine #11)
4-fold HA and 3-fold MF59 (vaccine #12).

In some embodiments, the oil-in-water emulsion adjuvant is a squalene-in-water emulsion adjuvant (e.g., MF59). In some embodiments, the standard-dose adjuvanted multivalent influenza vaccine comprises a squalene-in-water emulsion adjuvant having 9.75 mg squalene. In some embodiments, the standard-dose adjuvanted multivalent influenza vaccine comprises a squalene-in-water emulsion adjuvant having from about 5 mg to about 20 mg, from about 5 mg to about 15 mg, from about 5 mg to about 10 mg, from about 10 mg to about 20 mg, from about 10 mg to about 15 mg, or from about 15 mg to about 20 mg squalene. In some exemplary preferred embodiments, the quadrivalent vaccine composition may be vaccine #4, vaccine #6, vaccine #8, vaccine #9, or vaccine #11.

In some embodiments, the dose of the quadrivalent vaccine composition may range from 0.25 mL to 2.0 mL. In some particular exemplary embodiments, the dose of the quadrivalent vaccine composition may be 0.25 mL, 1.0 mL, 1.5 mL, or 2.0 mL In certain embodiments, the doses of the quadrivalent vaccine compositions with higher fold amounts of HA and/or MF59 may be higher. For example, in certain particular embodiments, the doses of vaccine #8 and/or vaccine #11 may range from 1.0 mL to 1.5 mL.

In some embodiments, one or more antigens are derived from influenza virus strains grown in egg. In some embodiments, one or more antigens are derived from influenza virus strains grown in cell culture. In some embodiments, all of the antigens are derived from influenza virus strains grown in cell culture.

In some embodiments, one or more antigens are from split viruses. In some embodiments, one or more antigens are from whole viruses. In some embodiments, one or more antigens are from attenuated whole viruses. In some embodiments, one or more antigens are from inactivated whole viruses. In some embodiments, one or more antigens are purified surface antigens (subunits). In some embodiments, all of the antigens are purified surface antigens (see, e.g., Example 1).

2. Methods for Inducing Immune Responses in Adults ≥65 Years of Age

By way of background, during the 2017-2018 season, rates of influenza-related hospitalizations and deaths increased dramatically among the elderly. Age-related immune dysfunction in older adults is believed to contribute to their vulnerability to influenza infection and may also compromise the effectiveness of conventional inactivated influenza vaccines. It is an aim of this disclosure to use the vaccine composition for safe and effective induction of immune responses in adults at least 65 years of age.

In some embodiments, a method for inducing an immune response in a human comprises administering to the human a vaccine composition comprising a) antigens from at least three different strains of influenza virus, preferably at least four different strains of influenza virus, and b) an oil-in-water emulsion adjuvant, wherein the amount of the oil-in-water emulsion adjuvant is greater than an amount of an oil-in-water emulsion adjuvant in a standard-dose adjuvanted multivalent influenza vaccine, and wherein the human is at least 65 years of age. In some embodiments, the total amount of the antigens in the vaccine compositions may be greater than a total amount of antigens in a standard-dose adjuvanted multivalent influenza vaccine.

In some embodiments, an administration of the vaccine composition according to this disclosure induces a higher seroconversion rate in a human compared to an administration of a standard-dose adjuvanted multivalent influenza vaccine (see example 1).

In some embodiments, an administration of a vaccine composition according to this disclosure enhances immunogenicity in a human at least 65 years of age, and preferably does not increase the incidence of unsolicited adverse events or systemic solicited adverse events compared to an administration of a standard-dose adjuvanted multivalent influenza vaccine.

For example, in some embodiments, an vaccine composition comprises 2-fold oil-in-water emulsion adjuvant, and administration of the vaccine composition does not increase the incidence of unsolicited adverse events or systemic solicited adverse events compared to an administration of a standard-dose adjuvanted multivalent influenza vaccine (see, e.g. Group 2 vs. Group 1 as shown in Example 1).

The immune responses raised by these methods generally include an antibody response, preferably a protective antibody response. Methods for assessing antibody responses, neutralizing capability and protection after influenza virus vaccination are well known in the art. Antibody responses are typically measured by hemagglutination inhibition, by microneutralization, by single radial immunodiffusion (SRID), and/or by single radial hemolysis (SRH). These assay techniques are well known in the art (see, e.g., Example 1).

Safety assessments are well known in the art. For example, they may include collection of all solicited and unsolicited adverse events (AEs) after administration of a vaccine composition according to this disclosure. Safety data may be analyzed descriptively, with the frequencies and percentages of subjects experiencing each AE presented for each symptom severity level (see, e.g., Example 1). In some embodiments, administration of a vaccine composition according to this disclosure in a human at least 65 years of age effectively induces immune responses, and preferably does not increase the incidence of unsolicited adverse events or systemic solicited adverse events compared to an administration of a standard-dose adjuvanted multivalent influenza vaccine.

Vaccine compositions according to this disclosure can be administered in various ways. In some embodiments, the vaccine composition may be administered to a human at least 65 years of age by intramuscular injection, subcutaneous delivery, intranasal delivery, oral delivery, intradermal delivery, transdermal delivery, transcutaneous delivery, or topical route. In some embodiments, a vaccine composition according to this disclosure is administered by intramuscular injection (see, e.g., Example 1).

Administration can be by a single-dose schedule or a multiple-dose schedule. Multiple doses may be used in a primary immunization schedule and/or in a booster immunization schedule. In a multiple dose schedule the various doses may be given by the same or different routes, e.g., a parenteral prime and mucosal boost, or a mucosal prime and parenteral boost. Multiple doses will typically be administered at least 1 week apart, e.g., about 2 weeks, about 3 weeks, about 4 weeks, about 6 weeks, about 8 weeks, about 10 weeks, about 12 weeks, or about 16 weeks. In some embodiments, the vaccine composition according to this disclosure is administered to a human in a single-dose schedule. In some embodiments, the vaccine composition according to this disclosure is administered to a human in a multiple-dose schedule.

Vaccines according to this disclosure may be administered to adults at substantially the same time as (e.g., during the same medical consultation or visit to a healthcare professional or vaccination center) other vaccines, e.g., at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A C W135 Y vaccine), a respiratory syncytial virus vaccine, or a pneumococcal conjugate vaccine. Administration at substantially the same time as a pneumococcal vaccine or a meningococcal vaccine is particularly useful in elderly patients, for example, those who are at least 65 years of age.

Similarly, vaccines according to this disclosure may be administered to adults at substantially the same time as (e.g., during the same medical consultation or visit to a healthcare professional) an antiviral compound, and in particular an antiviral compound active against influenza virus (e.g., oseltamivir and/or zanamivir). These antivirals include neuraminidase inhibitors, such as a (3R,4R,5S)-4-acetylamino-5-amino-3(1-ethylpropoxy)-1-cyclohexene-1-carboxylic acid or 5-(acetylamino)-4-[(aminoiminomethyl)-amino]-2,6-anhydro-3,4,5-trideoxy-D-glycero-D-galactonon-2-enonic acid, including esters thereof (e.g. the ethyl esters) and salts thereof (e.g. the phosphate salts). A preferred antiviral is (3R,4R,5S)-4-acetylamino-5-amino-3(1-ethylpropoxy)-1-cyclohexene-1-carboxylic acid, ethyl ester, phosphate (1:1), also known as oseltamivir phosphate (TAMIFLU™).

3. Kits and Methods for Producing Vaccine Compositions

In some embodiments, a method of producing the vaccine composition according this disclosure comprises admixing antigens from at least three different strains of influenza virus, preferably at least four different strains of influenza virus, and an oil-in-water emulsion adjuvant, wherein the amount of the oil-in-water emulsion adjuvant is greater than an amount of an oil-in-water emulsion adjuvant in a standard-dose adjuvanted multivalent influenza vaccine. In some embodiments, the total amount of the antigens in the vaccine compositions may be greater than a total amount of antigens in a standard-dose adjuvanted multivalent influenza vaccine.

Another aspect of this disclosure relates to preparations (e.g., kits) comprising a vaccine composition. The kit allows the adjuvant and the antigens to be kept separately until the time of use, which can be useful when using an oil-in-water emulsion adjuvant, such as MF59.

The components are physically separate from each other within a kit, and this separation can be achieved in various ways. For example, the two components may be in two separate containers, such as vials. The contents of the two vials can then be mixed, e.g., by removing the contents of one vial and adding them to the other vial, or by separately removing the contents of both vials and mixing them in a third container.

In one arrangement, one of the kit components is in a syringe and the other is in a container such as a vial. The syringe can be used (e.g., with a needle) to insert its contents into the second container for mixing, and the mixture can then be withdrawn into the syringe. The mixed contents of the syringe can then be administered to a human typically through a new sterile needle. Packing one component in a syringe eliminates the need for using a separate syringe for patient administration.

In another arrangement, the two kit components are held together but separately in the same syringe, e.g., a dual chamber syringe. When the syringe is actuated (e.g., during administration to an adult), the contents of the two chambers are mixed. This arrangement avoids the need for a separate mixing step at the time of use.

The kit components will generally be in aqueous form. In some embodiments, a component (typically the antigen component rather than the adjuvant component) is in dry form (e.g., in a lyophilized form), with the other component being in aqueous form. The two components can be mixed in order to reactivate the dry component and give an aqueous composition for administration to an adult. A lyophilized component will typically be located within a vial rather than a syringe. Dried components may include stabilizers such as lactose, sucrose or mannitol, as well as mixtures thereof. One possible arrangement uses an aqueous adjuvant component in a pre-filled syringe and a lyophilized antigen component in a vial.

Certain preferred embodiments of the present disclosure are summarized in the following paragraphs. This list is exemplary and not exhaustive of all of the embodiments provided by this disclosure.

Embodiment 1. A vaccine composition comprising antigens from at least four different strains of influenza virus and an oil-in-water emulsion adjuvant, wherein the amount of the oil-in-water emulsion adjuvant is greater than an amount of an oil-in-water emulsion adjuvant in a standard-dose adjuvanted multivalent influenza vaccine.

Embodiment 2. The vaccine composition of Embodiment 1, wherein the amount of the oil-in-water emulsion adjuvant is no greater than three-fold of an amount of an oil-in-water emulsion adjuvant in a standard-dose adjuvanted multivalent influenza vaccine.

Embodiment 3. The vaccine composition of Embodiment 1, wherein the amount of the oil-in-water emulsion adjuvant is no greater than two-fold of an amount of an oil-in-water emulsion adjuvant in a standard-dose adjuvanted multivalent influenza vaccine.

Embodiment 4. The vaccine composition of Embodiment 1, wherein the amount of the oil-in-water emulsion adjuvant is from two-fold to three-fold of an amount of an oil-in-water emulsion adjuvant in a standard-dose adjuvanted multivalent influenza vaccine.

Embodiment 5. The vaccine composition of any one of Embodiments 1-4, wherein the total amount of the antigens from the at least four different strains of influenza virus is from one-fold to four-fold of a total amount of antigens in a standard-dose adjuvanted multivalent influenza vaccine.

Embodiment 6. The vaccine composition of any one of Embodiments 1-5, wherein the total amount of the antigens from the at least four different strains of influenza virus is from one-fold to three-fold of a total amount of antigens in a standard-dose adjuvanted multivalent influenza vaccine.

Embodiment 7. The vaccine composition of any one of Embodiments 1-6, wherein the total amount of the antigens from the at least four different strains of influenza virus is from one-fold to two-fold of a total amount of antigens in a standard-dose adjuvanted multivalent influenza vaccine.

Embodiment 8. The vaccine composition of any one of Embodiments 1-7, wherein the standard-dose adjuvanted multivalent influenza vaccine comprises from about 5 mg to about 20 mg of oil-in-water emulsion adjuvant.

Embodiment 9. The vaccine composition of any one of Embodiments 1-8, wherein the standard-dose adjuvanted multivalent influenza vaccine comprises about 9.75 mg of oil-in-water emulsion adjuvant.

Embodiment 10. The vaccine composition of any one of Embodiments 1-9, wherein the antigens comprise hemagglutinin (HA).

Embodiment 11. The vaccine composition of any one of Embodiments 1-10, wherein the standard-dose adjuvanted multivalent influenza vaccine comprises from about 5 μg to about 30 μg hemagglutinin (HA) from each of the at least four different strains of influenza virus.

Embodiment 12. The vaccine composition of any one of Embodiments 1-11, wherein the standard-dose adjuvanted multivalent influenza vaccine comprises from about 10 μg to about 20 μg hemagglutinin (HA) from each of the at least four different strains of influenza virus.

Embodiment 13. The vaccine composition of any one of Embodiments 1-12, wherein the standard-dose adjuvanted multivalent influenza vaccine comprises about 15 ug hemagglutinin (HA) from each of the at least four different strains of influenza virus.

Embodiment 14. The vaccine composition of any one of Embodiments 1-13, wherein the at least four different strains of influenza virus are selected from the group consisting of influenza A, B, and/or C viruses.

Embodiment 15. The vaccine composition of any one of Embodiments 1-14, wherein the antigens from the at least four different strains of influenza virus are from two influenza A virus strains and two influenza B virus strains.

Embodiment 16. The vaccine composition of any one of Embodiments 14-15, wherein at least one of the four different strains of influenza virus is selected from: A/H1N1, A/H3N2, B/Yamagata, and B/Victoria.

Embodiment 17. The vaccine composition of any one of Embodiments 14-15, wherein at least two of the four different strains of influenza virus are selected from: A/H1N1, A/H3N2, B/Yamagata, and B/Victoria.

Embodiment 18. The vaccine composition of any one of Embodiments 14-15, wherein the at least four different strains of influenza virus are selected from: A/H1N1, A/H3N2, B/Yamagata, and B/Victoria.

Embodiment 19. The vaccine composition of any one of Embodiments 15-18, wherein the standard-dose adjuvanted multivalent influenza vaccine comprises about 15 μg hemagglutinin (HA) from each of the four different strains of influenza virus.

Embodiment 20. The vaccine composition of any one of Embodiments 1-19, wherein the oil-in-water emulsion adjuvant is a squalene-in-water emulsion adjuvant.

Embodiment 21. The vaccine composition of any one of Embodiments 1-20, wherein one or more antigens are derived from influenza virus strains grown in egg or cell culture.

Embodiment 22. The vaccine composition of any one of Embodiments 1-21, wherein one or more antigens are purified surface antigens.

Embodiment 23. The vaccine composition of any one of Embodiments 1-22, wherein the vaccine composition increases immunogenicity against at least 1, 2, 3, or 4 of the antigens in the vaccine composition.

Embodiment 24. The vaccine composition of any one of Embodiments 1-23 for use in a method of inducing an immune response in a human at least 65 years of age, comprising administering the vaccine composition to the human at least 65 years of age.

Embodiment 25. The vaccine composition of any one of Embodiments 1-23 for use in a method of enhancing immunogenicity in a human at least 65 years of age, comprising administering the vaccine composition to the human at least 65 years of age, wherein the administration of the vaccine composition induces enhanced immunogenicity in the human compared to an administration of a standard-dose adjuvanted multivalent influenza vaccine.

Embodiment 26. The vaccine composition of any one of Embodiments 1-23 for use in treating or preventing influenza infection in a human at least 65 years of age.

Embodiment 27. The vaccine composition of any one of Embodiments 1-23 for use in raising an immune response against influenza in a human at least 65 years of age.

Embodiment 28. The vaccine composition of any one of Embodiments 15-18, wherein the standard-dose adjuvanted multivalent influenza vaccine comprises at least about 15 μg hemagglutinin (HA) from each of the four different strains of influenza virus.

All of the claims in the claim listing are herein incorporated by reference into the specification in their entireties as additional embodiments.

### EXAMPLES

### Example 1: A Phase 1, Randomized, Observer Blind, Antigen and Adjuvant Dosage Finding Clinical Trial to Evaluate the Safety and Immunogenicity of an Adjuvanted, Trivalent Subunit Influenza Vaccine in Adults ≥65 Years of Age

The objective of this example is to assess the safety and immunogenicity of the MF59-adjuvanted trivalent influenza vaccine (aTIV; Fluad®) compared with modified aTIV formulations, in which the total amount of the antigens and/or the amount of MF59 exceeding that of the currently licensed standard-dose formulation.

Methods

1. Clinical Trial Design

This was a phase 1, randomized, observer blind, single centre, dosage-finding clinical trial of MF59-adjuvanted trivalent influenza vaccine (aTIV) in independently living elderly subjects. The trial was conducted in a phase 1 clinical trial unit in Berlin, Germany, and monitored by employees or representatives of Novartis Vaccines and, later, Seqirus Limited. The clinical trial was conducted in two parts. Part 1 comprised treatment Groups 1-4, and part 2 comprised treatments Groups 5-7. Enrolment was paused after day 8 for safety review by the Data Monitoring Committee (DMC), and part 2 of the trial continued only upon their recommendation. In part 1, subjects were randomized to one of the four treatment groups in a 1:1:1:1 ratio, with each subject administered one intramuscular (IM) injection in one deltoid. Group 1 received one dose of aTIV (containing 9.75 mg squalene and surfactants, referred to as MF59, as in standard aTIV); Group 2, one dose of aTIV formulated with a double dosage of MF59; Group 3, one dose of aTIV formulated with a double dosage of the antigens of the 3 influenza virus strains; and Group 4, two doses of aTIV, which constituted a double dosage of MF59 and a double dosage of the antigens of the 3 influenza virus strains (Table 1). During part 2, bilateral vaccinations were given to subjects randomized 1:1:1 to Groups 5-7. Group 5 received one dose of aTIV in the left deltoid and one dose of saline in the right deltoid; Group 6, one dose of aTIV formulated with an additional doubled dosage of MF59 in the left deltoid and one dose of saline in the right deltoid; and Group 7, one dose of aTIV in the left deltoid and a second, simultaneous dose of aTIV in the right deltoid.

The clinical trial was approved by the site institutional review board and was conducted in accordance with the Declaration of Helsinki and the International Council for Harmonisation Guideline for Good Clinical Practice. All participants gave written informed consent. To preserve the observer-blind design of the trial, the roles and responsibilities of the team members were prespecified.

2. Participants

Eligible subjects were men or women 65 years of age who were either healthy or had stable chronic illnesses. Individuals who had received any type of influenza vaccine (licensed or experimental) within the past 6 months or any other licensed vaccines (within 30 days for inactivated vaccines or 42 days for live vaccines) were excluded. The full list of inclusion and exclusion criteria can be found below.

Inclusion Criteria

Subjects who were eligible for inclusion in the clinical study met all of the following criteria:
1. Male and female subjects 65 years of age on the day of screening who were healthy or had chronic illnesses that were stable and well controlled.
2. Subjects assessed as mentally competent, who had given informed consent after the nature of the study had been explained according to local requirements.
3. In good health as determined by:
   a. Ability to live independently.
   b. Medical history.
   c. Physical examination.
   d. Clinical judgment of the Investigator.
4. Able to understand and complied with all study procedures and visits, and were able to complete an electronic diary.
5. Individuals who have had access to a working telephone and were able to receive periodic telephone calls.

TABLE 1
Vaccine treatment groups.

| | Part 1*: One | | | | Part 2*: Two bilateral | | |
|---|---|---|---|---|---|---|---|
| | intramuscular injection | | | | intramuscular injections^{†} | | |
| | Group 1 | Group 2 | Group 3 | Group 4 | Group 5 | Group 6 | Group 7 |
| | (n = 28) | (n = 28) | (n = 28) | (n = 27) | (n = 28) | (n = 28) | (n = 28) |
| MF59, | 9.75 | 19.5 | 9.75 | 19.5 | Left: 9.75 | Left: 29.25 | Left: 9.75 |
| mg/dosage | | | | | Right: 0^{‡} | Right: 0^{‡} | Right: 9.75 |
| HA antigen, | 45 | 45 | 90 | 90 | Left: 45 | Left: 45 | Left: 45 |
| μg/dosage | | | | | Right: 0^{‡} | Right: 0^{‡} | Right: 45 |

Abbreviations:
HA, haemagglutinin.
*Enrolment was done in two stages and paused after day 8 for safety review by the Data Monitoring Committee (DMC), after which the trial continued upon their recommendation.
^{†}Left and right deltoid.
^{‡}Saline injection.

Exclusion Criteria

Subjects who were eligible for the clinical study did not meet any of the following criteria:
1. Individuals who had received any type of influenza vaccine (licensed or experimental) within the past 6 months.
2. Individuals who had received any other licensed vaccines within 30 days (for inactivated vaccines) or 42 days (for live vaccines) prior to enrollment in this study.
3. Individuals who have had cancer except for:
   a. Benign localized skin cancer.
   b. Localized prostate cancer that has been clinically stable for years without treatment.
   c. Cancer in remission for 0 years (from end of cancer treatment).
4. Individuals with autoimmune disease (including rheumatoid arthritis).
5. Individuals with diabetes mellitus, type I.
6. Individuals with a body mass index (BMI) 8 or 35.
7. Asthma that was greater than mild in severity and/or has exacerbations more than 2 days per week.
8. Congestive heart failure with symptoms as severe as or more severe than dyspnea with short walks or climbing a single flight of stairs (for example, greater than New York Heart Association class 2).
9. History of progressive or severe neurologic disorders including but not limited to multiple sclerosis, Parkinson's disease, Guillain-Barré syndrome, amyotrophic lateral sclerosis, Creutzfeldt-Jakob disease, epilepsy disorders requiring medication for control, encephalitis, Alzheimer's and cerebrovascular accident (CVA).
10. Individuals who were hypersensitive to ovalbumin, chicken protein, chicken feathers, influenza viral protein, kanamycin and neomycin sulfate or any other component of the vaccines in study.
11. Individuals who have had a history of neurological symptoms or signs, or anaphylactic shock following administration of any vaccine.
12. Individuals who have had a known or suspected (or have a high risk of developing) impairment/alteration of immune function resulting from, for example:
   a. Receipt of immunosuppressive therapy (defined as follows) within the past 60 days and/or anticipated to receive immunosuppressive therapy at any point within 21 days of Visit 1.
      i. Cancer chemotherapy/radiotherapy
      ii. Systemic corticosteroids (i.e., 15 mg or greater per day of prednisone or equivalent)
      iii. Chronic use of inhaled/intranasal high potency corticosteroids (budesonide 800 μg per day or fluticasone 750 μg per day)
   b. Receipt of immunostimulants.
   c. Receipt of parenteral immunoglobulin preparation, blood products and/or plasma derivate within the past 3 months and for the full length of the study.
   d. Suspected or known human immunodeficiency virus (HIV) infection or HIV-related disease.
13. Individuals who have had a known or suspected history of drug or alcohol abuse.
14. Individuals who, within the past 12 months, have had laboratory confirmed influenza disease.
15. Individuals who, within the past 30 days have received any investigational agent.
16. Individuals who planned to receive another vaccine within 30 days of receipt of the study vaccination.
17. Individuals who, within the past 14 days, have experienced:
   a. Any acute disease including any worsening of underlying respiratory diseases such as asthma or chronic obstructive pulmonary disease (COPD).
   b. Infections requiring systemic antibiotic or antiviral therapy (chronic antibiotic therapy for urinary tract prophylaxis is acceptable).
18. Individuals who were taking part in another clinical study.
19. Individuals who were research staff directly involved with the clinical study or family members or household members of research staff. Research staff included an individual with direct or indirect contact with study subjects, or study site personnel who have had access to any study documents containing subject information. This would have included receptionists, persons scheduling appointments or making screening calls, regulatory specialists, laboratory technicians, etc.
20. Individuals with behavioral or cognitive impairment or a psychiatric condition that, in the opinion of the Investigator, may have interfered with the subject's ability to participate in the study.
21. Vulnerable subjects, e.g., subjects kept in detention, soldiers, employees of the Sponsor or a clinical research organization involved in this study.
22. Individuals who have had any condition which, in the opinion of the Investigator, might have interfered with the evaluation of the study objectives.

There may have been instances when individuals met all entry criteria except one that related to transient clinical circumstances (e.g., body temperature elevation or recent use of excluded medication or vaccine). Under these circumstances, a subject may have been considered eligible for study enrollment if the appropriate window for delay had passed, inclusion/exclusion criteria had been rechecked, and if the subject was confirmed to be eligible.

3. Vaccines and Procedures

The influenza virus strain composition was A/California/7/2009 (H1N1) pdm09-like virus, A/Texas/50/2012 (H3N2)-like virus, and B/Massachusetts/2/2012-like virus, as determined by the World Health Organisation (WHO) for trivalent vaccines contemporaneous to the timing of the study. Groups 1 and 5 received a vaccine identical to licensed aTIV (Fluad®, Seqirus Inc., Cambridge, Mass.), containing 9.75 mg of MF59 and 45 μg of HA antigen. Other groups received varying amounts of MF59 (9.75, 19.5, or 29.25 mg/dosage) and the HA antigen (45 or 90 μg/dosage; Table 1).

Vaccines were given via intramuscular injection in the deltoid on Day 1. Groups 1˜4 received a single injection in the nondominant arm. Groups 5 and 6 received active vaccine in the left arm and saline in the right arm. Group 7 received active vaccine in both arms.

Blood for serologic analysis was collected on study days 1, 8, 22, and 181. On study days 1-7, subjects made a daily record of adverse events (AEs) in an electronic diary. All subjects received training in the rationale and use of the electronic diary prior to start of the trial.

4. Endpoints

The primary endpoint was based on immune response as assessed using the hemagglutination inhibition (HI) and microneutralization (MN) assays on Day 22. Associated analyses included the geometric mean titres (GMTs) on days 1 and day 22, the geometric mean ratios (GMRs), defined as the GMT on Day 22 over the GMT on Day 1, the percentage of subjects achieving seroconversion on Day 22, and the percentage of subjects achieving a ≥4-fold rise in MN titre on Day 22 from Day 1. Seroconversion was defined as a pre-vaccination HI titre <10 and a post-vaccination HI titre ≥40 or a pre-vaccination HI titre ≥10 and a minimum 4-fold rise in post-vaccination HI antibody titre.

Secondary endpoints included analysis of the primary immune response endpoints on Days 8 and 181.

Safety assessments included collection of all solicited and unsolicited adverse events (AEs), the latter including serious adverse events (SAEs), new onset of chronic disease (NOCD), and adverse events of special interest (AESIs). AEs were reported verbally during study visits or telephone safety assessments. Solicited AEs were recorded using the electronic diary on study days 1-7 as absent, mild, moderate, or severe. Unsolicited AEs were recorded as mild, moderate or severe. All unsolicited AEs were categorized according to Medical Dictionary for Regulatory Activities (MedDRA, Version 17.0) preferred terms and assessed for relationship to study vaccine. Patients recorded occurrence of solicited local and systemic AEs using an electronic diary for 7 days following the dose of vaccine, and telephone calls 14 days after the study vaccine dose were used to collect solicited AEs persisting beyond 7 days and unsolicited AEs occurring thereafter. After study day 28, only AEs that necessitated a non-scheduled physician visit, medical attention, or study withdrawal; SAEs: NOCD; and AESI were collected until study completion. All AEs were monitored until resolution or until a cause identified, if the AE became chronic. The below predefined list of AESI medical concepts and MedDRA preferred terms were provided to the study investigator.

Prespecified Adverse Events of Special Interest

The following prespecified list of medical concepts were given to study investigators and defined according to the Medical Dictionary for Regulatory Activities (MedDRA, Version 17.0) preferred terms:
Neuroinflammatory disorders: optic neuritis, uveitis, multiple sclerosis, demyelinating disease, transverse myelitis, Guillain-Barre syndrome, myasthenia gravis, encephalitis, ADEM, neuritis, Bell's palsy.
Musculoskeletal and connective tissue disorders: rheumatoid arthritis, juvenile rheumatoid arthritis, polymyalgia rheumatica, psoriatic arthropathy, ankylosing spondylitis, systemic lupus erythematosis, cutaneous lupus, Sjogren's syndrome, scleroderma, dermatomyositis, polymyositis, mixed connective tissue disease, reactive arthritis
Vasculidites: temporal arteritis, Wegener's granulomatosis, mixed connective tissue disease
Gastrointestinal disorders: Crohn's disease, ulcerative colitis, inflammatory bowel disease (non-specific), celiac disease, autoimmune hepatitis, primary sclerosing cholangitis, primary biliary cirrhosis
Renal disorders: glomerulonephritis, nephritis, renal vasculitis
Cardiac disorders: carditis, pericarditis, myocarditis, cardiomyopathy
Skin disorders: psoriasis, vitiligo, Raynaud's phenomenon, erythema nodosum, autoimmune bullous skin diseases, Stevens-Johnson syndrome
Hematologic disorders: autoimmune hemolytic anemia, idiopathic thrombocytopenic purpura, antiphospholipid syndrome, pernicious anemia
Metabolic disorders: autoimmune thyroiditis, Grave's or Basedow's disease, Hashimoto thyroiditis, insulin-dependent diabetes mellitus, Addison's disease
Others: sarcoidosis

In general, the scales used to assess the severity of solicited AE were based on recommendations of the US Food and Drug Administration (FDA) [17]. The severity of solicited local AEs, including injection site erythema, swelling, and induration was categorized according to linear measurement: 26-50 mm, 51-100 mm, and >100 mm. Injection site pain and systemic reactions (except fever) occurring up to 7 days after vaccination were categorized as “mild,” “moderate,” or “severe.” Fever was defined as body temperature 38.0° C. The use of antipyretics and analgesics was summarized by frequency of use.

5. Statistical Analysis

The full analysis set included all subjects who were randomized, received a study vaccination, and provided at least one immunogenicity finding. The per protocol set included everyone from the full analysis set who was not excluded for a protocol deviation or withdrawal of consent. All immunogenicity assessments were performed using the per protocol set, and the primary objectives were also assessed using the full analysis set as a sensitivity analysis. The safety set included all subjects exposed to study vaccine who provided records of post-vaccination adverse events and/or reactogenicity. The sample size was selected to show a significant difference (2-sided α=0.10) between the aTIV and modified formulation groups assuming the true response in the modified formulation group was a 3-fold higher GMT providing 80% to 95% power for the individual paired comparisons. No adjustments for multiplicity were made as the study was exploratory in nature and all comparisons were intended to be descriptive.

The GMTs and the associated two-sided 95% confidence intervals (CIs) were constructed by exponentiation (base 10) of the least square means of the logarithmically transformed (base 10) antibody titres. Adjusted estimates of GMTs at Day 22 and their associated 95% CIs were determined using analysis of covariance (ANCOVA) with the following terms for covariates: treatment group and log-transformed prevaccination antibody titer. Comparisons (for both HI and MN) between groups were based on the adjusted GMTs measured at Day 22 and the associated two-sided 95% confidence intervals. The analysis of GMR relative to Day 1 was also computed using this ANCOVA model. Furthermore, comparisons (for both HI and MN) between relevant groups were analysed using a model with antigen and MF59 dosage factors as additional fixed effects. The primary endpoints based on the percentage of subjects with seroconversion or significant increase in antibody HI titres (Day 22), 4-fold increase in MN titre (Day 22), and the associated Clopper-Pearson two-sided 95% CIs were computed for each influenza strain and for each vaccination group at Day 22. The percentages of subjects with HI titres ≥1:40, ≥1:110, ≥1:160, and ≥1:330 on Days 8, 22, and 181 and associated Clopper-Pearson two-sided 95% CIs were provided. Individual treatment differences (as percentages) along with the two-sided 95% CIs using the Miettinen-Nurminen algorithm [18].

Equal dosing in one or two arms was also compared using the same model as for the primary analysis comparing Groups 4 and 7 (19.5 mg of MF59 in both). No multiplicity adjustment was performed as the study was exploratory in nature. All HI and MN titres below the lower limit of detection (i.e., 10) were set to half that limit (i.e., 5).

Safety data were analysed descriptively, with the frequencies and percentages of subjects experiencing each AE presented for each symptom severity level.

Results

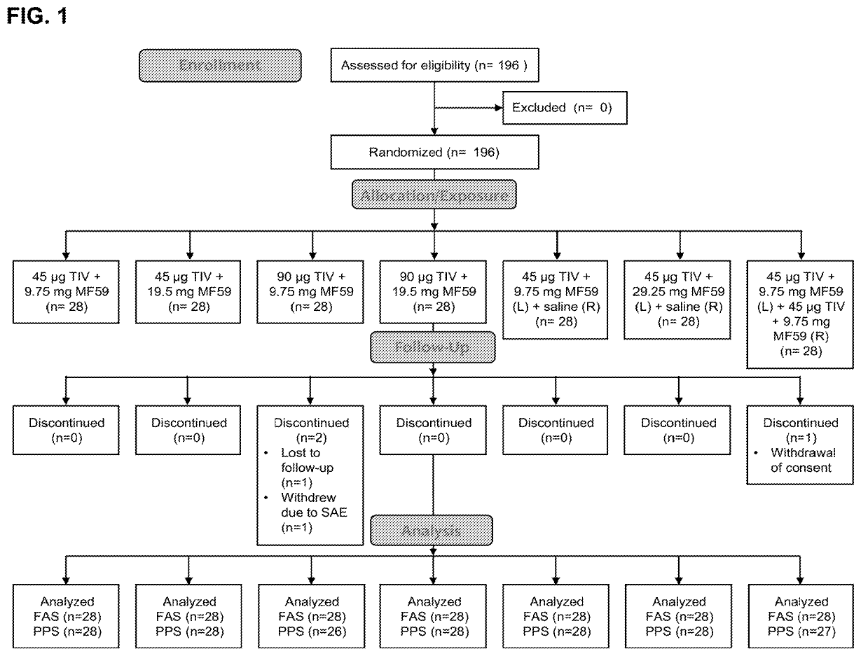

The full analysis set comprised 196 subjects randomly assigned to the seven treatment groups who received vaccine; the per protocol set included 195 subjects (FIG. 1). Mean age in the total population was 70.5 years and was similar between treatment groups (Table 2). All subjects were non-Hispanic whites, and approximately half were female, although the proportion of women in different treatment groups varied from 32.1% to 71.4%. The mean BMI was 26.77 kg/m².

TABLE 2
Baseline demographics and characteristics.

| | Part 1: One | | | | Part 2: Two | | | |
|---|---|---|---|---|---|---|---|---|
| | intramuscular injection | | | | bilateral intramuscular injections | | | |
| | Group 1 | Group 2 | Group 3 | Group 4 | Group 5 | Group 6 | Group 7 | Total |
| | (n = 28) | (n = 28) | (n = 28) | (n = 27) | (n = 28) | (n = 28) | (n = 28) | (N = 195) |
| Mean age, | 69.7 | 71.5 | 70.3 | 71.6 | 70.0 | 70.9 | 69.5 | 70.5 |
| years | | | | | | | | |
| Female sex, | 14 (50.0) | 20 (71.4) | 13 (46.4) | 12 (44.4) | 11 (39.3) | 12 (42.9) | 9 (32.1) | 91 (46.7) |
| n (%) | | | | | | | | |
| White, not | 28 (100) | 28 (100) | 28 (100) | 27 (100) | 28 (100) | 28 (100) | 28 (100) | 195 (100) |
| Hispanic | | | | | | | | |
| Mean weight ± | 75.1 ± 9.1 | 72.1 ± 12.2 | 76.6 ± 11.6 | 77.3 ± 12.2 | 77.6 ± 16.1 | 74.8 ± 12.3 | 82.4 ± 13.2 | 76.5 ± 12.7 |
| SD, kg | | | | | | | | |
| Mean BMI ± | 26.7 ± 3.0 | 26.6 ± 2.9 | 26.9 ± 3.4 | 26.9 ± 3.8 | 26.8 ± 3.8 | 25.8 ± 3.5 | 27.6 ± 3.0 | 26.8 ± 3.3 |
| SD, kg/m² | | | | | | | | |

Abbreviations:
BMI, body mass index;
SD, standard deviation.

1. Immune Response

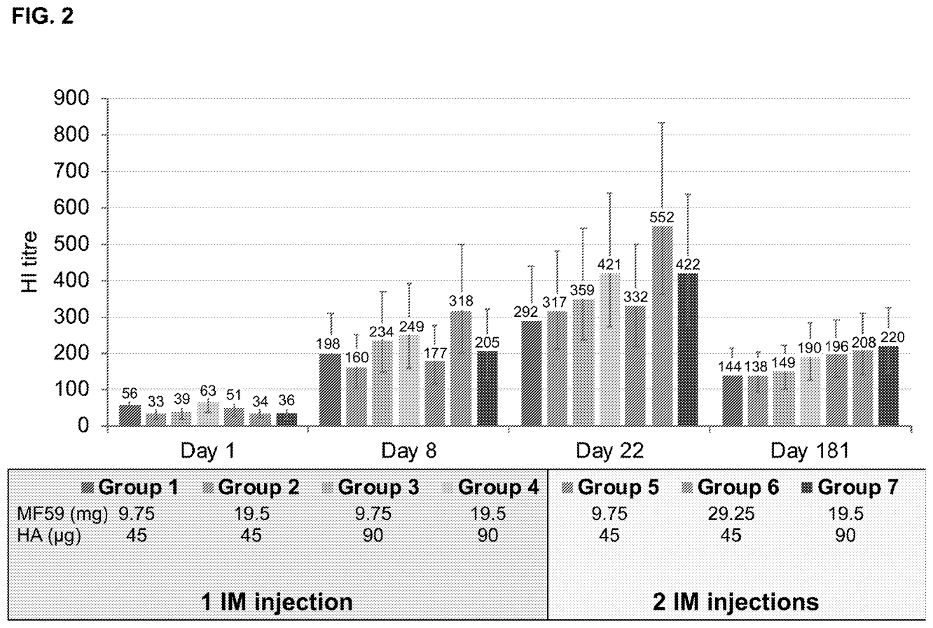

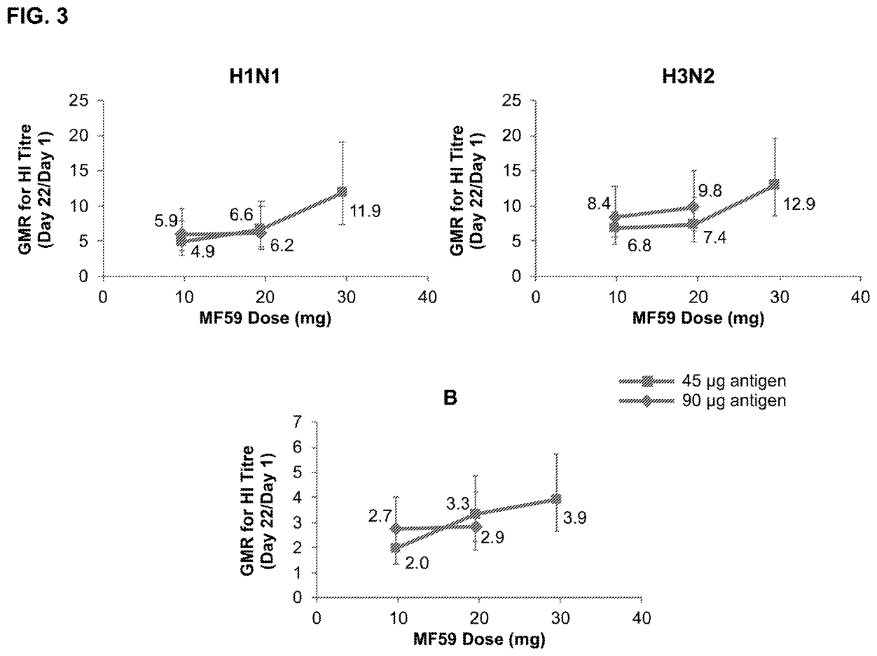

Immune response patterns as evaluated with HI GMTs and GMRs across all groups were established by Day 8 and persisted throughout the study period (FIG. 2, Table 3). Day 22 GMTs and GMRs for all influenza strains showed a trend favouring higher dosages of MF59 (FIG. 3). The overall highest GMTs and GMRs on Day 22 were observed in Group 6 for A/H3N2: GMT, 552.3 (95% CI 364.8 to 836.1); GMR, 12.9 (8.5 to 19.5). Group 6 also had the highest GMTs and GMRs for A/H1N1 (GMT, 382.2 [237.5 to 615.0]; GMR, 11.9 [7.4 to 19.1]) and B strain (GMT, 54.1 [36.9 to 79.4]; GMR, 3.9 [2.7 to 5.7]) on Day 22.

On Day 8, the highest overall HI GMT (281.1 [172.0 to 459.2]) and GMR (8.6 [5.3 to 13.9]) were observed in Group 6 for A/H1N1, and the next highest GMT (317.5 [201.0 to 501.8]) and GMR (8.0 [5.1 to 12.5]) were for A/H3N2 in the same group, which received the 29.25 mg dosage of MF59. Group 6 also exhibited the highest GMT (42.7 [31.0 to 58.7]) and GMR (3.1 [2.2 to 4.2]) for B strain on Day 8.

By Day 181, HI titres had diminished but the pattern remained generally similar to Day 8 titres. The highest overall GMT (220.3 [148.4 to 326.8]) and GMR (5.1 [3.4 to 7.5]) on Day 181 was for A/H3N2 in Group 7, which received 9.75 mg MF59 and 90 μg of HA antigen. The highest GMT (160.9 [108.0 to 239.8]) and GMR for A/H1N1 (4.9 [3.3 to 7.3]) was in Group 5, which received the equivalent of the standard dosage of MF59 and HA (9.75 mg and 45 μg, respectively), and the highest GMT (26.0 [19.3 to 35.1]) and GMR (1.9 [1.4 to 2.5]) for B strain was in Group 7. The Group 6 GMRs on Day 181 were 4.5 (3.0 to 6.8) for A/H1N1, 4.8 (3.2 to 7.2) for A/H3N2, and 1.7 (1.3 to 2.4) for B strain.

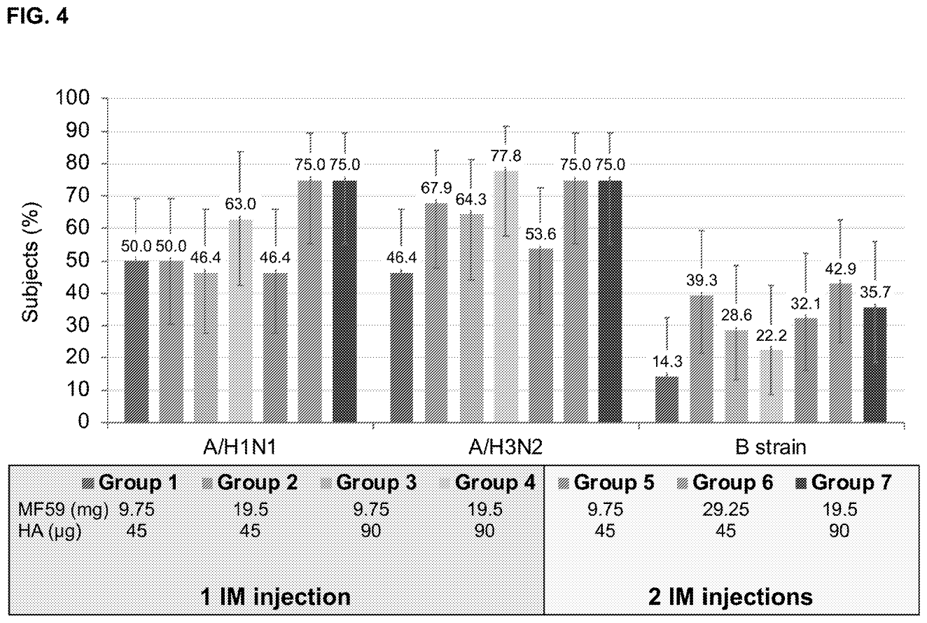

As shown in FIG. 4, seroconversion rates were also highest in Group 4 for A/H3N2 (77.8% [95% CI 57.7 to 91.4]), followed by Group 2 (67.9% [47.6 to 84.1]) and Group 3 (64.3% [44.1 to 81.4]). Seroconversion rates for A/H1N1 were highest in Group 4 (63.0% [42.4 to 80.6]), whereas for B strain seroconversion occurred most often in Group 2 (39.3% [21.5 to 59.4]).

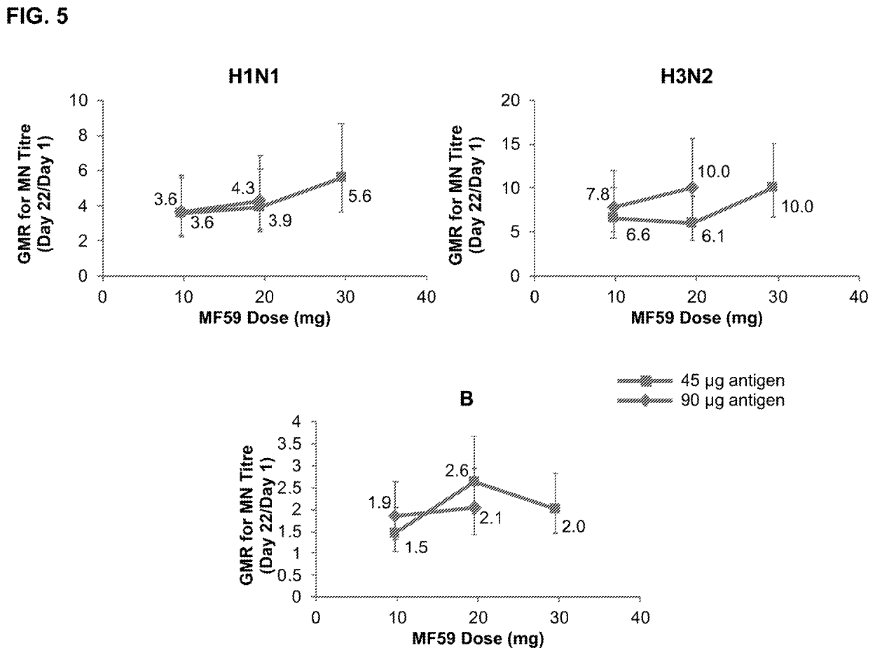

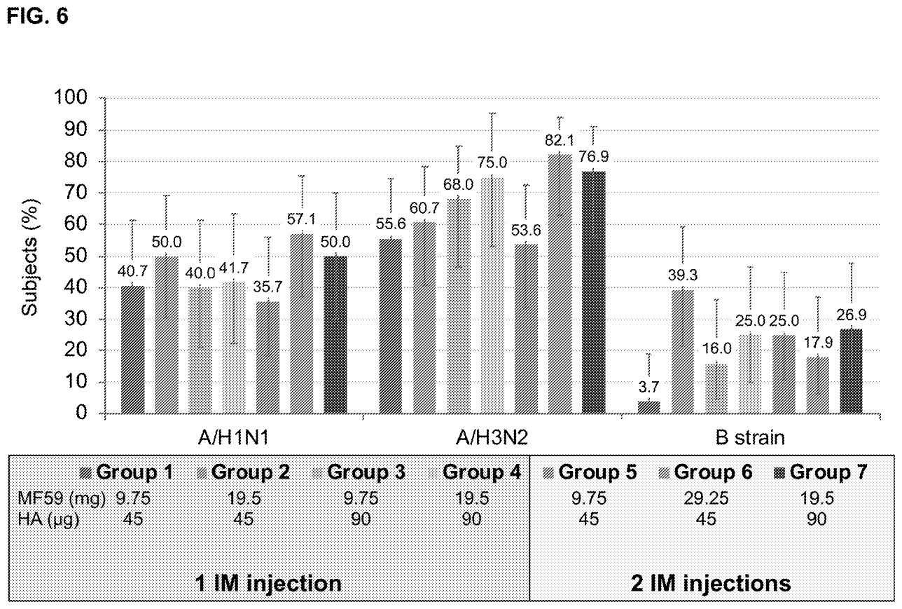

The immunogenicity results based on the MN assay at Days 8, 22, and 181 were similar to the HI results (Table 4), with greater immunogenicity associated with higher MF59 dosage, except for B strain (FIG. 5). The highest overall MN assay results were observed for A/H3N2 in Group 4 on Day 8 (GMT 191.3 [117.1 to 312.3]; GMR 7.2 [4.4 to 11.8]) and Day 22 (GMT 265.0 [170.2 to 412.5]; GMR 10.0 [6.4 to 15.6]) and in Group 7 on Day 181 (GMT 141.0 [91.7 to 216.8]; GMR 5.3 [3.4 to 8.1]) (Table 4). The highest proportion of subjects achieving a 4-fold increase in MN titres was in Group 6 for A/H3N2 (82.1% [63.1 to 93.9]) and A/H1N1 (57.1% [37.2 to 75.5]) and Group 2 for B strain (39.3% [21.5 to 59.4]) (FIG. 6).

TABLE 3
Geometric mean titres and geometric mean ratios for A/H1N1, A/H3N2, and B strains determined by
hemagglutination inhibition in the per protocol set.*

| | Part 1: | | | | Part 2: Two | | |
|---|---|---|---|---|---|---|---|
| | One intramuscular injection | | | | bilateral intramuscular injections | | |
| | Group 1 | Group 2 | Group 3 | Group 4 | Group 5 | Group 6 | Group 7 |
| MF59/HA, mg/μg | (n = 28) | (n = 28) | (n = 28) | (n = 27) | (n = 28) | (n = 28) | (n = 28) |
| per dose | 9.75/45 | 19.5/45 | 9.75/90 | 19.5/90 | 9.75/45 | 29.25/45 | 9.75/90 |
| A/H1N1 | | | | | | | |
| GMT Day 1 | 34.5 (19.5 to | 40.0 (20.5 to | 34.0 (18.5 to | 33.8 (21.3 to | 44.7 (24.1 to | 21.3 (12.7 to | 24.1 (14.0 to |
| (baseline) | 60.8) | 77.9) | 62.7) | 53.7) | 82.9) | 35.5) | 41.5) |
| (95% CI)^{†} | | | | | | | |
| GMT Day 8 | 135.8 (84.0 | 118.3 (73.2 | 167.4 (102.7 | 165.5 (101.5 | 132.9 (82.1 | 281.1 (172.0 | 196.3 (121.3 |
| (95% CI)^{‡} | to 219.4) | to 191.3) | to 272.8) | to 269.8) | to 215.1) | to 459.2) | to 317.6) |
| GMR Day 8/ | 4.2 (2.6 to | 3.6 (2.3 to | 5.1 (3.1 to | 5.1 (3.1 to | 4.1 (2.5 to | 8.6 (5.3 to | 6.1 (3.8 to |
| Day 1 (95% CI)^{‡} | 6.7) | 5.9) | 8.2) | 8.3) | 6.6) | 13.9) | 9.8) |
| GMT Day 22 | 156.5 (97.5 | 213.0 (132.6 | 191.5 (119.3 | 198.4 (122.6 | 238.0 (148.1 | 382.2 (237.5 | 297.0 (184.8 |
| (95% CI)^{§} | to 251.1) | to 342.0) | to 307.3) | to 321.2) | to 382.6) | to 615.0) | to 477.2) |
| GMR Day 22/ | 4.9 (3.0 to | 6.6 (4.1 to | 5.9 (3.7 to | 6.2 (3.8 to | 7.4 (4.6 to | 11.9 (7.4 to | 9.2 (5.7 to |
| Day 1 (95% CI)^{§} | 7.8) | 10.6) | 9.5) | 10.0) | 11.9) | 19.1) | 14.8) |
| GMT Day 181 | 69.8 (46.9 to | 98.2 (65.9 to | 73.8 (49.3 to | 74.8 (49.9 to | 160.9 (108.0 | 146.8 (97.8 | 100.1 (67.2 |
| (95% CI)^{‡} | 103.8) | 146.2) | 110.7) | 112.2) | to 239.8) | to 220.5) | to 149.2) |
| GMR Day 181/ | 2.1 (1.4 to | 3.0 (2.0 to | 2.3 (1.5 to | 2.3 (1.5 to | 4.9 (3.3 to | 4.5 (3.0 to | 3.1 (2.1 to |
| Day 1 (95% CI)^{‡} | 3.2) | 4.5) | 3.4) | 3.4) | 7.3) | 6.8) | 4.6) |
| A/H3N2 | | | | | | | |
| GMT Day 1 | 55.8 (30.2 to | 33.2 (19.3 to | 38.5 (20.0 to | 62.6 (34.6 to | 50.6 (26.7 to | 33.6 (18.7 to | 35.8 (19.1 to |
| (baseline) | 103.4) | 57.1) | 74.1) | 113.4) | 95.9) | 60.4) | 66.9) |
| (95% Cl)^{†} | | | | | | | |
| GMT Day 8 | 198.4 (126.5 | 160.2 (102.2 | 233.8 (148.0 | 248.5 (157.1 | 177.5 (113.3 | 317.5 (201.0 | 204.6 (130.5 |
| (95% CI)^{‡} | to 311.0) | to 251.2) | to 369.3) | to 393.2) | to 278.1) | to 501.8) | to 320.7) |
| GMR Day 8/ | 4.6 (2.9 to | 3.7 (2.4 to | 5.2 (3.3 to | 5.8 (3.6 to | 4.1 (2.6 to | 8.0 (5.1 to | 4.7 (3.0 to |
| Day 1 (95% CI)^{‡} | 7.2) | 5.8) | 8.2) | 9.2) | 6.5) | 12.5) | 7.4) |
| GMT Day 22 | 292.3 (193.0 | 317.2 (209.5 | 359.3 (237.5 | 421.1 (275.8 | 331.8(219.2 | 552.3 (364.8 | 421.8(278.7 |
| (95% CI)^{§} | to 442.5) | to 480.2) | to 543.7) | to 643.0) | to 502.1) | to 836.1) | to 638.3) |
| GMR Day 22/ | 6.8 (4.5 to | 7.4 (4.9 to | 8.4 (5.5 to | 9.8 (6.4 to | 7.7 (5.1 to | 12.9 (8.5 to | 9.8 (6.5 to |
| Day 1 (95% CI)^{§} | 10.3) | 11.2) | 12.7) | 15.0) | 11.7) | 19.5) | 14.9) |
| GMT Day 181 | 144.0 (97.0 | 138.0 (93.0 | 149.2 (99.9 | 190.0 (127.0 | 196.1 (132.2 | 207.9 (139.1 | 220.3 (148.4 |
| (95% CI)^{‡} | to 213.7) | to 204.9) | to 223.0) | to 284.3) | to 290.9) | to 310.7) | to 326.8) |
| GMR Day 181/ | 3.3 (2.2 to | 3.2(2.1 to | 3.4 (2.3 to | 4.4 (2.9 to | 4.5 (3.0 to | 4.8 (3.2 to | 5.1 (3.4t0 |
| Day 1 (95% CI)^{‡} | 4.9) | 4.7) | 5.1) | 6.5) | 6.7) | 7.2) | 7.5) |
| B strain | | | | | | | |
| GMT Day 1 | 15.6 (10.4 to | 12.8 (9.2 to | 14.1 (9.9 to | 18.0 (12.5 to | 12.6 (8.7 to | 13.1 (9.4 to | 11.6 (8.4 to |
| (baseline) | 23.4) | 17.8) | 20.3) | 26.0) | 18.4) | 18.3) | 16.1) |
| (95% CI)^{†} | | | | | | | |
| GMT Day 8 | 27.2 (19.9 to | 35.1 (25.6 to | 32.4 (23.5 to | 32.8 (23.8 to | 34.8 (25.5 to | 42.7 (31.0 to | 33.9 (24.7 to |
| (95% CI)^{‡} | 37.3) | 48.0) | 44.6) | 45.2) | 47.7) | 58.7) | 46.4) |
| GMR Day 8/ | 2.0 (1.4 to | 2.5 (1.9 to | 2.3 (1.7 to | 2.4 (1.7 to | 2.5 (1.8 to | 3.1 (2.2 to | 2.5 (1.8 to |
| Day 1 (95% CI)^{‡} | 2.7) | 3.5) | 3.1) | 3.3) | 3.5) | 4.2) | 3.4) |
| GMT Day 22 | 27.1 (18.5 to | 45.9 (31.3 to | 37.7 (25.7 to | 39.5 (26.6 to | 45.5 (31.0 to | 54.1 (36.9 to | 41.4 (28.2 to |
| (95% CI)^{§} | 39.8) | 67.3) | 55.4) | 58.4) | 66.8) | 79.4) | 60.8) |
| GMR Day 22/ | 2.0 (1.3 to | 3.3 (2.3 to | 2.7 (1.9 to | 2.9 (1.9 to | 3.3 (2.2 to | 3.9 (2.7 to | 3.0 (2.0 to |
| Day 1 (95% CI)^{§} | 2.9) | 4.9) | 4.0) | 4.2) | 4.8) | 5.7) | 4.4) |
| GMT Day 181 | 18.8 (13.9 to | 21.2 (15.7 to | 21.4 (15.8 to | 24.7 (18.2 to | 23.5 (17.4 to | 23.9 (17.6 to | 26.0 (19.3 to |
| (95% CI)^{‡} | 25.4) | 28.6) | 29.0) | 33.6) | 31.7) | 32.4) | 35.1) |
| GMR Day 181/ | 1.4 (1.0 to | 1.5 (1.1 to | 1.6 (1.1 to | 1.8 (1.3 to | 1.7 (1.3 to | 1.7 (1.3 to | 1.9 (1.4 to |
| Day 1 (95% CI)^{‡} | 1.8) | 2.1) | 2.1) | 2.4) | 2.3) | 2.4) | 2.5) |

Abbreviations:
CI, confidence interval;
GMR, geometric mean ratio;
GMT, geometric mean titre;
HA, haemagglutinin;
HI, hemagglutination inhibition;
MN, microneutralization.
*Both GMT/GMR and CI of log10 (titre) were calculated using ANCOVA with log10 (baseline) and group as covariates. The GMT and CI of Day 1 (baseline) was calculated descriptively.
^{†}Day 1 GMT values shown are for the primary endpoint analysis using the PPS. GMRs for Day 8/Day 1 and Day 181/Day 1 may be based on slightly different GMT values due to slight differences in the number of subjects with serum available for analysis.
^{‡}Secondary endpoint.
^{§}Primary endpoint.

TABLE 4
Geometric mean titres and geometric mean ratios for A/H1N1, A/H3N2, and B strains determined by
microneutralization assay in the per protocol set.*

| | | | | | Part 2: Two | | |
|---|---|---|---|---|---|---|---|
| | Part 1: One intramuscular injection | | | | bilateral intramuscular injections* | | |
| | Group 1 | Group 2 | Group 3 | Group 4 | Group 5 | Group 6 | Group 7 |
| MF59/HA, mg/μg | (n = 28) | (n = 28) | (n = 28) | (n = 27) | (n = 28) | (n = 28) | (n = 28) |
| per dose | 9.75/45 | 19.5/45 | 9.75/90 | 19.5/90 | 9.75/45 | 29.25/45 | 9.75/90 |
| A/H1N1 | | | | | | | |
| GMT Day 1 | 10.7 (7.6 to | 12.8 (8.3 to | 10.6 (7.4 to | 9.6 (7.0 to | 13.6 (9.6 to | 9.1 (6.9 to | 9.5 (7.2 to |
| (baseline) | 14.9) | 19.9) | 15.0) | 13.0) | 19.4) | 12.2) | 12.5) |
| (95% Cl)^{†} | | | | | | | |
| GMT Day 8 (95% | 32.3 (20.9 to | 28.7 (18.7 to | 38.2 (24.1 to | 38.3 (24.1 to | 29.0 (18.9 to | 37.3 (23.7 to | 38.6 (24.8 to |
| CI)^{‡} | 50.0) | 44.1) | 60.6) | 60.8) | 44.6) | 58.7) | 60.3) |
| GMR Day 8/ Day | 3.0 (1.9 to | 2.6 (1.7 to | 3.5 (2.2 to | 3.5 (2.2 to | 2.7 (1.7 to | 3.3 (2.1 to | 3.5 (2.3 to |
| 1 (95% CI)^{‡} | 4.6) | 4.0) | 5.4) | 5.6) | 4.1) | 5.1) | 5.5) |
| GMT Day 22 | 38.7 (24.8 to | 42.1 (27.2 to | 38.8 (24.4 to | 46.0 (28.7 to | 41.7 (26.7 to | 60.1 (38.8 to | 53.9 (33.9 to |
| (95% CI)^{§} | 60.4) | 65.2) | 61.6) | 73.7) | 65.3) | 93.1) | 85.6) |
| GMR Day 22/ | 3.6 (2.3 to | 3.9 (2.5 to | 3.6 (2.3 to | 4.3 (2.7 to | 3.9 (2.5 to | 5.6 (3.6 to | 5.0 (3.1 to |
| Day 1 (95% CI)^{§} | 5.6) | 6.1) | 5.7) | 6.8) | 6.1) | 8.6) | 7.9) |
| GMT Day 181 | 18.9 (13.7 to | 22.7 (16.6 to | 17.1 (12.2 to | 21.1 (15.1 to | 29.1 (21.2 to | 28.9 (20.9 to | 25.9 (18.7 to |
| (95% CI)^{‡} | 26.1) | 31.2) | 24.1) | 29.7) | 39.9) | 39.8) | 35.9) |
| GMR Day 181/ | 1.7 (1.3 to | 2.1 (1.5 to | 1.6 (1.1 to | 2.0 (1.4 to | 2.7 (2.0 to | 2.7 (1.9 to | 2.4 (1.7 to |
| Day 1 (95% CI)^{‡} | 2.4) | 2.9) | 2.2) | 2.7) | 3.7) | 3.7) | 3.3) |
| A/H3N2 | | | | | | | |
| GMT Day 1 | 32.6 (18.9 to | 25.3 (15.3 to | 22.3 (12.2 to | 34.6 (18.1 to | 32.8 (18.8 to | 19.0 (11.3 to | 22.9 (12.5 to |
| (baseline) | 56.1) | 41.9) | 40.9) | 66.2) | 57.2) | 32.2) | 41.8) |
| (95% CI)^{†} | | | | | | | |
| GMT Day 8 (95% | 114.2 (71.9 | 85.4 (54.3 to | 139.3 (85.3 | 191.3 (117.1 | 87.3 (55.5 to | 155.9 (98.2 | 118.1(73.7 to |
| CI^{)‡} | to 181.2) | 134.4) | to 227.3) | to 312.3) | 137.4) | to 247.7) | 189.0) |
| GMR Day 8/ | 4.3 (2.7 to | 3.2 (2.0 to | 5.2 (3.2 to | 7.2 (4.4 to | 3.3 (2.1 to | 6.4 (4.0 to | 4.4 (2.8 to |
| Day 1 (95% CI)^{‡} | 6.9) | 5.1) | 8.3) | 11.8) | 5.2) | 10.0) | 7.1) |
| GMT Day 22 | 174.4 (114.9 | 160.2 (106.5 | 205.0 (133.0 | 265.0 (170.2 | 191.0 (126.8 | 264.3 (175.3 | 237.1 (155.1 |
| (95% CI)^{§} | to 264.6) | to 241.1) | to 316.1) | to 412.5) | to 287.7) | to 398.4) | to 362.5) |
| GMR Day 22/ | 6.6 (4.4 to | 6.1 (4.0 to | 7.8 (5.0 to | 10.0 (6.4 to | 7.2 (4.8 to | 10.0 (6.6 to | 9.0 (5.9 to |
| Day 1 (95% CI)^{§} | 10.0) | 9.1) | 12.0) | 15.6) | 10.9) | 15.1) | 13.8) |
| GMT Day 181 | 82.8 (54.2 to | 68.1 (45.0 to | 83.1 (53.1 to | 112.3 (71.7 | 102.6 (67.7 | 123.2 (80.7 | 141.0 (91.7 to |
| (95% CI)^{‡} | 126.3) | 103.1) | 130.1) | to 175.8) | to 155.4) | to 188.1) | 216.8) |
| GMR Day 181/ | 3.1 (2.0 to | 2.6 (1.7 to | 3.1 (2.0 to | 4.2 (2.7 to | 3.8 (2.5 to | 4.6 (3.0 to | 5.3 (3.4 to |
| Day 1 (95% CI)^{‡} | 4.7) | 3.9) | 4.9) | 6.6) | 5.8) | 7.1) | 8.1) |
| B strain | | | | | | | |
| GMT Day 1 | 6.5 (5.5 to | 5.9 (5.1 to | 6.4 (5.4 to | 6.3 (5.3 to | 6.9 (5.7 to | 5.9 (5.0 to | 6.1 (5.1 to |
| (baseline) | 7.6) | 6.9) | 7.7) | 7.6) | 8.4) | 7.0) | 7.3) |
| (95% CI)^{†} | | | | | | | |
| GMT Day 8 (95% | 8.4 (6.5 to | 11.0 (8.5 to | 9.1 (7.0 to | 9.4 (7.2 to | 9.3 (7.2 to | 10.2 (7.9 to | 11.7 (9.0 to |
| CI)^{‡} | 10.9) | 14.1) | 12.0) | 12.4) | 12.0) | 13.3) | 15.2) |
| GMR Day 8/ | 1.3 (1.0 to | 1.7 (1.4 to | 1.4 (1.1 to | 1.5 (1.1 to | 1.5 (1.1 to | 1.6 (1.2 to | 1.9 (1.4 to |
| Day 1 (95% CI)^{‡} | 1.7) | 2.2) | 1.9) | 2.0) | 1.9) | 2.1) | 2.4) |
| GMT Day 22 | 9.2 (6.6 to | 16.6 (11.9 to | 11.7 (8.2 to | 13.0 (9.1 to | 13.0 (9.4 to | 12.8 (9.2 to | 16.7 (11.9 to |
| (95% CI)^{§} | 12.9) | 23.0) | 16.5) | 18.5) | 18.1) | 17.7) | 23.4) |
| GMR Day 22/ | 1.5 (1.1 to | 2.6 (1.9 to | 1.9 (1.3 to | 2.1 (1.4 to | 2.1 (1.5 to | 2.0 (1.5 to | 2.7 (1.9 to |
| Day 1 (95% CI)^{§} | 2.0) | 3.7) | 2.6) | 2.9) | 2.9) | 2.8) | 3.7) |
| GMT Day 181 | 6.8 (5.5 to | 8.2 (6.7 to | 7.7 (6.1 to | 7.7 (6.1 to | 8.4 (6.8 to | 8.0 (6.5 to | 10.0 (8.1 to |
| (95% CI)^{‡} | 8.5) | 10.2) | 9.7) | 9.7) | 10.4) | 10.0) | 12.5) |
| GMR Day 181/ | 1.1 (0.9 to | 1.3 (1.1 to | 1.2 (1.0 to | 1.2 (1.0 to | 1.3 (1.1 to | 1.3 (1.0 to | 1.6 (1.3 to |
| Day 1 (95% CI)^{‡} | 1.3) | 1.6) | 1.5) | 1.5) | 1.7) | 1.6) | 2.0) |

Abbreviations:
CI, confidence interval;
GMR, geometric mean ratio;
GMT, geometric mean titre;
HA, haemagglutinin;
HI, hemagglutination inhibition;
MN, microneutralization.
*Both GMT/GMR and CI of log10 (titre) were calculated using ANCOVA with log10 (baseline) and group as covariates. The GMT and CI of Day 1 (baseline) was calculated descriptively.
^{†}Day 1 GMT values shown are for the primary endpoint analysis using the PPS. GMRs for Day 8/Day 1 and Day 181/Day 1 may be based on slightly different GMT values due to slight differences in the number of subjects with serum available for analysis.
^{‡}Secondary endpoint.
^{§}Primary endpoint.

2. Safety

Table 5 summarizes adverse events during the study. Across study groups, between 25.0% (Group 7) and 67.9% (Group 4) of subjects experienced a solicited AE. As was expected, the incidence of solicited, localized AEs increased with increasing dosage of MF59 and with HA antigen at the 19.5 mg dosage of MF59 but not at the standard dosage of 9.75 mg Table 6). The most frequent local solicited AE was pain, which was reported by 35.7% of subjects in Group 6. Groups 3 and 6 had the highest incidence of localized erythema (17.9% in both), and the highest incidence of induration occurred in Group 6 (14.3%). The majority of solicited AEs were assessed as either mild or moderate in severity.

Surprisingly, in certain treatment group, the frequency of systemic AEs did not increase dramatically with increasing dosages of MF59 (see, e.g., Group 2 vs. Group 1). The most frequent systemic solicited AE was fatigue, which occurred in 35.7% subjects from Group 4 and 25.0% of subjects from Group 6 (Table 6). The majority of solicited systemic AEs resolved within 72 hours, with only isolated events of fatigue, headache, loss of appetite, and malaise with longer durations. The highest reported analgesic use was reported in Groups 2 and 7, with no evident group-related pattern.

Neither the incidence nor severity of solicited AEs increased among subjects who received the double dose of aTIV split into 2 injections, one in each arm (Group 7), compared with subjects who received the same dose in a single injection in one arm (Group 4). The majority of the solicited adverse events reported were mild to moderate in severity, and only 2 solicited AEs (1 case each of injection site hematoma and pain at injection site) were ongoing after 7 days after vaccination.

TABLE 5
Summary of subjects with solicited and unsolicited adverse events.

| | | | | | Part 2: | | |
|---|---|---|---|---|---|---|---|
| | Part 1: One | | | | Two bilateral intramuscular | | |
| | intramuscular injection | | | | injections* | | |
| | Group 1 | Group 2 | Group 3 | Group 4 | Group 5 | Group 6 | Group 7 |
| Solicited AE safety | 28 | 28 | 28 | 28 | 28 | 28 | 28 |
| set, no. subjects | | | | | | | |
| Solicited AEs,* n (%) | 12 (42.9) | 10 (35.7) | 13 (46.4) | 19 (67.9) | 11 (39.3) | 16 (57.1) | 7 (25.0) |
| Unsolicited AE safety | 14 | 16 | 17 | 13 | 17 | 21 | 17 |
| set, no. subject^{†} | | | | | | | |
| Possibly/probably | 1 (7.1) | 2 (12.5) | 0 | 3 (23.1) | 1 (5.9) | 1 (4.8) | 1 (5.9) |
| related AEs, n (%) | | | | | | | |
| Medically attended | 7 (50.0) | 10 (62.5) | 10 (58.8) | 6 (46.2) | 7 (41.2) | 7 (33.3) | 10 (58.8) |
| AEs, n (%) | | | | | | | |
| Withdrawal due to | 0 | 0 | 1 (5.9) | 0 | 0 | 0 | 0 |
| AE, n (%) | | | | | | | |
| New onset chronic | 0 | 1 (6.3) | 0 | 0 | 3 (17.6) | 0 | 0 |
| disease, n (%) | | | | | | | |
| AEs of special | 0 | 0 | 0 | 0 | 0 | 0 | 0 |
| interest, n (%) | | | | | | | |
| SAEs not including | 1 (7.1) | 1 (6.3) | 1 (5.9) | 1 | 0 | 0 | 0 |
| death, n (%) | | | | | | | |
| Possibly/probably | 0 | 0 | 0 | 0 | 0 | 0 | 0 |
| related SAEs, n | | | | | | | |
| Deaths, n | 0 | 0 | 0 | 0 | 0 | 0 | 0 |

*Solicited AEs were reported from Day 1 through Day 7. For vaccination regimens administered in both arms (left and right) only the maximum severity event was counted; each subject was then counted only once per event.
^{†}The unsolicited safety set comprised the subject population who reported an unsolicited AE.

TABLE 6
Local and systemic solicited adverse events.

| | | | | | Two bilateral intramuscular | | |
|---|---|---|---|---|---|---|---|
| | One intramuscular injection | | | | injections* | | |
| | Group 1 | Group 2 | Group 3 | Group 4 | Group 5 | Group 6 | Group 7 |
| Adverse event, n (%) | (n = 28) | (n = 28) | (n = 28) | (n = 28) | (n = 28) | (n = 28) | (n = 28) |
| Any solicited AE* | 12 (42.9) | 10 (35.7) | 13 (46.4) | 19 (67.9) | 11 (39.3) | 16 (57.1) | 7 (25.0) |
| Any local solicited AE | 6 (21.4) | 6 (21.4) | 7 (25.0) | 11 (39.3) | 8 (28.6) | 14 (50.0) | 6 (21.4) |
| Ecchymosis | 1 (3.6) | 0 | 0 | 1 (3.6) | 1 (3.6) | 1 (3.6) | 0 |
| Erythema | 2 (7.1) | 2 (7.1) | 5 (17.9) | 4 (14.3) | 3 (10.7) | 5 (17.9) | 1 (3.6) |
| Induration | 2 (7.1) | 2 (7.1) | 1 (3.6) | 1 (3.6) | 2 (7.1) | 4 (14.3) | 1 (3.6) |
| Pain | 3 (10.7) | 6 (21.4) | 3 (10.7) | 7 (25.0) | 6 (21.4) | 10 (35.7) | 6 (21.4) |
| Swelling | 2 (7.1) | 1 (3.6) | 2 (7.1) | 4 (14.3) | 2 (7.1) | 6 (21.4) | 1 (3.6) |
| Any systemic AE | 7 (25.0) | 6 (21.4) | 7 (25.0) | 14 (50.0) | 8 (28.6) | 10 (35.7) | 5 (17.9) |
| Arthralgia | 0 | 0 | 1 (3.6) | 2 (7.1) | 1 (3.6) | 1 (3.6) | 1 (3.6) |
| Chills | 0 | 0 | 0 | 0 | 0 | 1 (3.6) | 2 (7.1) |
| Diarrhoea | 0 | 0 | 0 | 0 | 2 (7.1) | 1 (3.6) | 0 |
| Fatigue | 4 (14.3) | 4 (14.3) | 2 (7.1) | 10 (35.7) | 3 (10.7) | 7 (25.0) | 2 (7.1) |
| Headache | 2 (7.1) | 1 (3.6) | 2 (7.1) | 4 (14.3) | 2 (7.1) | 4 (14.3) | 3 (10.7) |
| Loss of appetite | 0 | 1 (3.6) | 1 (3.6) | 1 (3.6) | 0 | 2 (7.1) | 1 (3.6) |
| Malaise | 2 (7.1) | 1 (3.6) | 1 (3.6) | 2 (7.1) | 2 (7.1) | 4 (14.3) | 1 (3.6) |
| Myalgia | 2 (7.1) | 1 (3.6) | 3 (10.7) | 4 (14.3) | 0 | 2 (7.1) | 3 (10.7) |
| Nausea | 0 | 0 | 1 (3.6) | 0 | 0 | 2 (7.1) | 1 (3.6) |

*Solicited AEs were reported from Day 1 through Day 7. Reported is the number of subjects who had at least one AE (i.e. multiple similar events reported within an individual is counted only once). For vaccination regimens administered in both arms (left and right) only the maximum severity event was counted; each subject was then counted only once per event.

Across the groups, between 13 (Group 4) and 21 (Group 6) subjects in each group reported an unsolicited AE (Table 5). Among this subpopulation, AEs considered possibly or probably related to vaccine occurred in 4.8% of subjects in Group 6; 5.9% of subjects in Groups 5 and 7; and 7.1%, 12.5%, and 23.1% of subjects in Groups 1, 2, and 4, respectively. No subjects in Group 3 reported a possibly or probably related AE. These included isolated reports of nasopharyngitis, injection site pain or warmth, arthralgia, headache, and rash in single subjects. No treatment group-related trends were observed.

No deaths were reported during the study. In total, four SAEs (all considered unrelated to vaccination) were reported, including coronary heart disease, coronary artery disease, metastasis to bone within cup-syndrome, and carotid artery stenosis. One SAE (metastasis to bone within cup-syndrome) resulted in subject withdrawal. No AEs of special interest were reported. Four events of new onset of chronic disease were reported, none of which were prespecified: hypertension in Group 2 and abnormal glucose tolerance test, osteoporosis, and ligament sprain in Group 5.

### CONCLUSIONS

The current formulation of aTIV, containing 45 μg of hemagglutinin (15 μg/strain) and MF59 containing 9.75 mg of squalene, has been sold in Europe since 1997 and in the Unites States since 2016. The addition of MF59 to seasonal influenza vaccines increases the immunogenicity, persistence, and breadth of protection in children and older adults [7, 19]. aTIV has been shown to be more effective than nonadjuvanted vaccines in adults 65 years and older [15, 16, 20]. Recently, a seasonal quadrivalent influenza vaccine containing MF59 was shown to have superior clinical efficacy in children 6 through 23 months of age, compared to a non-adjuvanted vaccine [21]. This is the first study to assess the immunogenicity and safety of dosages of hemagglutinin and MF59 exceeding that of the currently licensed formulation.

In this study involving subjects 65 years of age, dosages of MF59 up to three times higher and hemagglutinin dosages up to 2-fold higher than the currently licensed formulation of aTIV were administered. The vaccine containing the 3-fold higher dosage of MF59 was associated with the highest post-vaccination GMT measured by HI at Day 22 and, accounting for baseline, GMR, for all three strains. A 2-fold increase in the amount of hemagglutinin resulted in similar immunogenicity measured by GMR, compared with a two-fold increase in MF59. The trends in greater immune response after vaccination with a 3-fold dosage of MF59, as assessed by either HI or MN assays, were apparent 8 days after immunization.

The study vaccines containing higher dosages of MF59 and influenza antigen were generally well tolerated compared to the currently licensed formulation of aTIV. The incidence of solicited AEs was higher in subjects who received the highest dosages of MF59, but there were no apparent trends in terms of the incidence of unsolicited AEs. The solicited adverse reactions associated with the higher dosages of MF59, including pain, swelling, erythema, induration, were mostly mild to moderate in severity. This pattern of increased local reactogenicity is consistent with other studies of aTIV in subjects 65 years and older [7, 22]. MF59 typically increases local reactogenicity in older individuals, and the increased pain may reflect the enhanced immune response due to the adjuvant. Increasing dosages of MF59 did not appear to result in an increase in systemic solicited adverse events, with the possible exception of fatigue. At standard dosages of MF59 (9.75 mg), increasing HA antigen from 45 to 90 μg did not lead to an increased frequency of solicited AEs, but at higher dosages (19.5 mg) of MF59, the incidence of solicited AEs nearly doubled with 90 μg of antigen. The increase in influenza-specific immune response attributed to the higher dosages of MF59 also resulted in an increase in local reactogenicity, but without any other serious safety concerns, including potentially immune mediated diseases.

The results from the study showed a dosage and response effect of MF59 that was comparable to that of antigen and which continued to increase without evidence of plateau. This study demonstrated the relative impact of increasing amounts of MF59 and influenza antigens on the immunogenicity and safety after vaccination in older adults 65 years of age.

### Example 2: Safety and Immunogenicity of Adjuvanted Quadrivalent Subunit Influenza Vaccines in Adults ≥65 Years of Age

Various adjuvanted quadrivalent subunit influenza vaccines are prepared (FIG. 7). For example, vaccine #6 contains 2-fold MF59 as well as 2-fold HA antigens compared to a standard-dose quadrivalent influenza vaccine. In contrast, vaccine #8 contains 2-fold MF59 as well as 4-fold HA antigens compared to a standard-dose quadrivalent influenza vaccine. The volume of the vaccine can optionally be adjusted. For example, vaccine #11 contains 3-fold MF59 as well as 3-fold HA antigens per 1.5 mL dose, compared to a standard-dose quadrivalent influenza vaccine. The final volume of vaccine #11 can optionally be adjusted to 1.0 mL, resulting in altered MF59 and antigen concentrations.

In this study, subjects 65 years are randomized to receive the vaccines shown in FIG. 7. The objective is to study the immunogenicity and safety of quadrivalent influenza vaccines accordingly to this disclosure, for inducing immune responses in adults at least 65 years of age.

The primary and second endpoints analyses are based on immune response assessed following the procedures in Example 1. Safety assessments include collection of all solicited and unsolicited adverse events following the procedures in Example 1.

This study shows that quadrivalent influenza vaccines accordingly to this disclosure are safe and effective at inducing immune responses in adults at least 65 years of age.

### Example 3: Safety, Tolerability, and Immunogenicity of Adjuvanted Quadrivalent Subunit Influenza Vaccines Purpose/Objective of Study

The objective of this study is to evaluate the safety, tolerability and immunogenicity of several formulations of quadrivalent influenza vaccine, including vaccines comprising cell-derived hemagglutinin (HA) antigens in doses ranging from 15 to 60 μg per strain and an MF59 adjuvant in doses ranging from 9.8 to 29.4 mg of squalene per dose (a standard, a double, or a triple dose).

Study Design

The study is a randomized, controlled, observer-blind trial being conducted in approximately 720 healthy older adults. In this study, six investigational formulations are tested: two formulations with 15 μg HA per strain and different amount of MF59 (9.8 mg and 29.4 mg, respectively), one formulation with 30 μg of HA and 19.6 mg of MF59, one formulation with 45 μg HA per strain and 29.4 mg of MF59, and two formulations with 60 μg of HA per strain and 9.8 mg and 19.6 mg of MF59 per dose, respectively. Two licensed vaccines—Quadrivalent Subunit Inactivated Cell-derived Influenza Vaccine (QIVc, Flucelvax, Seqirus) and High Dose Quadrivalent Inactivated Egg-derived Influenza Vaccine (Fluzone HD, SP)—are used as comparators.

Study Recruitment

Firstly, a lead-in cohort of approximately 80 healthy adults 50 to 64 years of age (10 subjects per group) is recruited to evaluate the safety and tolerability of all proposed regimens. Following a confirmation of acceptable safety and tolerability profile, the recruitment of subjects 65 years of age and above is initiated. Approximately 640 older adult subjects (80 subjects per group) are recruited during the second part of the study.

Study Schedule

The study consists of a vaccination phase in which subjects are vaccinated at baseline (Day 1) and followed for reactogenicity, safety and immunogenicity assessment for 28 days, and a follow-up phase in which subjects are followed for safety and antibody persistence (from Day 29 to Day 181).

Study Groups

Subjects in the study are randomized into eight different groups of approximately 90 healthy subjects each (including 10 subjects 50-64 years and 80 subjects 65 years and above) in a 1:1:1:1:1:1:1:1 ratio as follows:
15HA/1MF59 group: approximately 90 subjects receiving one dose of investigational vaccine (15 μg of HA for each of four vaccine strains and a standard dose of MF59 (9.8 mg)) at Day 1.
15HA/3MF59 group: approximately 90 subjects receiving one dose of investigational vaccine (15 μg of HA for each of four vaccine strains and a triple dose of MF59 (29.4 mg)) at Day 1.
30HA/2MF59 group: approximately 90 subjects receiving one dose of investigational vaccine (30 μg of HA for each of four vaccine strains and a double dose of MF59 (19.6 mg)) at Day 1.
45HA/3MF59 group: approximately 90 subjects receiving one dose of investigational vaccine (45 μg of HA for each of four vaccine strains and a triple dose of MF59 (29.4 mg)) at Day 1.
60HA/1 MF59 group: approximately 90 subjects receiving one dose of investigational vaccine (60 μg of HA for each of four vaccine strains and a standard dose of MF59 (9.8 mg)) at Day 1.
60HA/2MF59 group: approximately 90 subjects receiving one dose of investigational vaccine (60 μg of HA for each of four vaccine strains and a double dose of MF59 (19.6 mg)) at Day 1.
QIVc group: approximately 90 subjects receiving one dose of the licensed quadrivalent cell-derived influenza vaccine (QIVc) at Day 1.
QIV HD group: approximately 90 subjects receiving one dose of the licensed high-dose quadrivalent egg-derived influenza vaccine (HD QIV) at Day 1.

Safety

Safety is assessed by collection of immediate post-vaccination AEs (at 30 minutes after vaccination), solicited local and systemic adverse events (within 7 days post-vaccination), unsolicited adverse events (within 28 days post-vaccination), serious adverse events and AEs of special interest (during the entire study period), and by physical examination.

Immunogenicity is assessed using the following serological assays:
Haemagglutinin Inhibition Assay (HI) for homologous vaccine strains, using cell-derived target virus, is performed in all subjects on serum samples collected on Days 1, 29 and 181.
Microneutralization Assay (MN) for homologous vaccine strains, using cell-derived target virus, is performed in all subjects on serum samples collected on Days 1, 29 and 181.
Optionally, HI assay for selected heterologous influenza strains, using cell-derived target virus, is performed on serum samples collected on Days 1 and 29.

The exploratory assay package optionally includes, but is not limited to, the listed assays.

Scoring/Assessment

A multi-criteria decision-making approach, a desirability model, is used to identify which investigational vaccine formulation has the most desirable immune response 28 days after vaccination and safety profile comparing to the currently licensed vaccines. An overall desirability index is obtained by taking a weighted geometric mean of the four desirability indexes associated to each of the four vaccine strains and a desirability index associated with vaccine tolerability.

What is claimed is:

1. A vaccine composition comprising antigens from at least three different strains of influenza virus and an oil-in-water emulsion adjuvant, wherein the amount of the oil-in-water emulsion adjuvant is greater than an amount of an oil-in-water emulsion adjuvant in a standard-dose adjuvanted multivalent influenza vaccine.

2. A vaccine composition comprising antigens from at least four different strains of influenza virus and an oil-in-water emulsion adjuvant, wherein the amount of the oil-in-water emulsion adjuvant is greater than an amount of an oil-in-water emulsion adjuvant in a standard-dose adjuvanted multivalent influenza vaccine.

3. The vaccine composition of any one of claims 1 and 2, wherein the amount of the oil-in-water emulsion adjuvant is no greater than three-fold of an amount of an oil-in-water emulsion adjuvant in a standard-dose adjuvanted multivalent influenza vaccine.

4. The vaccine composition of any one of claims 1 and 2, wherein the amount of the oil-in-water emulsion adjuvant is no greater than two-fold of an amount of an oil-in-water emulsion adjuvant in a standard-dose adjuvanted multivalent influenza vaccine.

5. The vaccine composition of any one of claims 1 and 2, wherein the amount of the oil-in-water emulsion adjuvant is from two-fold to three-fold of an amount of an oil-in-water emulsion adjuvant in a standard-dose adjuvanted multivalent influenza vaccine.

6. The vaccine composition of any one of claims 1-5, wherein the total amount of the antigens from the at least three or four different strains of influenza virus is from one-fold to four-fold of a total amount of antigens in a standard-dose adjuvanted multivalent influenza vaccine.

7. The vaccine composition of any one of claims 1-6, wherein the total amount of the antigens from the at least three or four different strains of influenza virus is from one-fold to three-fold of a total amount of antigens in a standard-dose adjuvanted multivalent influenza vaccine.

8. The vaccine composition of any one of claims 1-7, wherein the total amount of the antigens from the at least three or four different strains of influenza virus is from one-fold to two-fold of a total amount of antigens in a standard-dose adjuvanted multivalent influenza vaccine.

9. The vaccine composition of any one of claims 1-8, wherein the standard-dose adjuvanted multivalent influenza vaccine comprises from about 5 mg to about 20 mg of oil-in-water emulsion adjuvant.

10. The vaccine composition of any one of claims 1-9, wherein the standard-dose adjuvanted multivalent influenza vaccine comprises about 9.75 mg of oil-in-water emulsion adjuvant.

11. The vaccine composition of any one of claims 1-10, wherein the antigens comprise hemagglutinin (HA).

12. The vaccine composition of any one of claims 1-11, wherein the standard-dose adjuvanted multivalent influenza vaccine comprises from about 5 μg to about 30 μg hemagglutinin (HA) from each of the strains of influenza virus.

13. The vaccine composition of any one of claims 1-12, wherein the standard-dose adjuvanted multivalent influenza vaccine comprises from about 10 μg to about 20 μg hemagglutinin (HA) from each of the strains of influenza virus.

14. The vaccine composition of any one of claims 1-13, wherein the standard-dose adjuvanted multivalent influenza vaccine comprises about 15 ug hemagglutinin (HA) from each of the strains of influenza virus.

15. The vaccine composition of any one of claims 1 and 3-14, wherein the antigens from the at least three different strains of influenza virus are from two influenza A virus strains and one influenza B virus strain.

16. The vaccine composition of claim 15, wherein the standard-dose adjuvanted multivalent influenza vaccine comprises 15 μg hemagglutinin (HA) from each of the at least three different strains of influenza virus.

17. The vaccine composition of any one of claims 15 and 16, wherein the antigens from the at least three different strains of influenza virus induce an immune response against A/H1N1, AH3N2, and/or B virus strains.

18. The vaccine composition of any one of claims 2-14, wherein the antigens from the at least four different strains of influenza virus are from two influenza A virus strains and two influenza B virus strains.

19. The vaccine composition of claim 18, wherein the standard-dose adjuvanted multivalent influenza vaccine comprises 15 μg hemagglutinin (HA) from each of the at least four different strains of influenza virus.

20. The vaccine composition of any one of claims 1-19, wherein the at least three or four different strains of influenza virus are selected from the group consisting of influenza A, B, and/or C viruses.

21. The vaccine composition of any one of claims 1, 3-14, and 19, wherein the at least three different strains of influenza virus are selected from the group consisting of A/California/7/2009 (H1N1) pdm09-like virus, A/Texas/50/2012 (H3N2)-like virus, and B/Massachusetts/2/2012-like virus.

22. The vaccine composition of any one of claims 1-21, wherein the oil-in-water emulsion adjuvant is a squalene-in-water emulsion adjuvant.

23. The vaccine composition of any one of claims 1-22, wherein one or more antigens are derived from influenza virus strains grown in egg or cell culture.

24. The vaccine composition of any one of claims 1-23, wherein one or more antigens are purified surface antigens.

25. A method of inducing an immune response in a human, comprising administering to the human a vaccine composition comprising antigens from at least three or four different strains of influenza virus and an oil-in-water emulsion adjuvant, wherein the amount of the oil-in-water emulsion adjuvant is greater than an amount of an oil-in-water emulsion adjuvant in a standard-dose adjuvanted multivalent influenza vaccine, and wherein the human is at least 65 years of age.

26. The method of claim 25, wherein the amount of the oil-in-water emulsion adjuvant is no greater than three-fold of an amount of an oil-in-water emulsion adjuvant in a standard-dose adjuvanted multivalent influenza vaccine.

27. The method of claim 25, wherein the amount of the oil-in-water emulsion adjuvant is no greater than two-fold of an amount of an oil-in-water emulsion adjuvant in a standard-dose adjuvanted multivalent influenza vaccine.

28. The method of claim 25, wherein the amount of the oil-in-water emulsion adjuvant is from two-fold to three-fold of an amount of an oil-in-water emulsion adjuvant in a standard-dose adjuvanted multivalent influenza vaccine.

29. The method of any one of claims 25-28, wherein the total amount of the antigens from the at least three or four different strains of influenza virus is from one-fold to three-fold of a total amount of antigens in a standard-dose adjuvanted multivalent influenza vaccine.

30. The method of any one of claims 25-29, wherein the total amount of the antigens from the at least three or four different strains of influenza virus is from one-fold to three-fold of a total amount of antigens in a standard-dose adjuvanted multivalent influenza vaccine.

31. The method of any one of claims 25-30, wherein the total amount of the antigens from the at least three or four different strains of influenza virus is from one-fold to two-fold of a total amount of antigens in a standard-dose adjuvanted multivalent influenza vaccine.

32. The method of any one of claims 25-31, wherein the standard-dose adjuvanted multivalent influenza vaccine comprises from about 5 mg to about 20 mg of oil-in-water emulsion adjuvant.

33. The method of any one of claims 25-32, wherein the standard-dose adjuvanted multivalent influenza vaccine comprises about 9.75 mg of oil-in-water emulsion adjuvant.

34. The method of any one of claims 25-33, wherein the antigens comprise hemagglutinin (HA).

35. The method of any one of claims 25-34, wherein the standard-dose adjuvanted multivalent influenza vaccine comprises from about 5 μg to about 30 μg hemagglutinin (HA) from each of the strains of influenza virus.

36. The method of any one of claims 25-35, wherein the standard-dose adjuvanted multivalent influenza vaccine comprises from about 10 μg to about 20 μg hemagglutinin (HA) from each of the strains of influenza virus.

37. The method of any one of claims 25-36, wherein the standard-dose adjuvanted multivalent influenza vaccine comprises about 15 ug hemagglutinin (HA) from each of the strains of influenza virus.

38. The method of any one of claims 25-37, wherein the antigens from the at least three different strains of influenza virus are from two influenza A virus strains and one influenza B virus strain.

39. The method of claim 38, wherein the standard-dose adjuvanted multivalent influenza vaccine comprises 15 μg hemagglutinin (HA) from each of the at least three different strains of influenza virus.

40. The method of any one of claims 38 and 39, wherein the antigens from the at least three different strains of influenza virus induce an immune response against A/H1N1, A/H3N2, and/or B virus strains.

41. The method of any one of claims 38-40, wherein the at least three different strains of influenza virus are selected from the group consisting of A/California/7/2009 (H1N1) pdm09-like virus, A/Texas/50/2012 (H3N2)-like virus, and B/Massachusetts/2/2012-like virus.

42. The method of any one of claims 25-37, wherein the antigens from the at least four different strains of influenza virus are from two influenza A virus strains and two influenza B virus strains.

43. The method of claim 42, wherein the standard-dose adjuvanted multivalent influenza vaccine comprises 15 μg hemagglutinin (HA) from each of the at least four different strains of influenza virus.

44. The method of any one of claims 25-43, wherein the oil-in-water emulsion adjuvant is a squalene-in-water emulsion adjuvant.

45. The method of any one of claims 25-44, wherein one or more antigens are derived from influenza virus strains grown in egg or cell culture.

46. The method of any one of claims 25-45, wherein one or more antigens are purified surface antigens.

47. The method of any one of claims 25-46, wherein the administration of the vaccine composition induces a higher seroconversion rate in the human compared to an administration of a standard-dose adjuvanted multivalent influenza vaccine.

48. The method of claim 47, wherein the higher seroconversion rate is 67.9%.

49. The method of any one of claims 25-48, wherein the administration of the vaccine composition induces an immune response in the human without increase in unsolicited adverse events compared to an administration of a standard-dose adjuvanted multivalent influenza vaccine.

50. The method of any one of claims 25-49, wherein preferably the administration of the vaccine composition induces an immune response in the human without increase in systemic solicited adverse events compared to an administration of a standard-dose adjuvanted multivalent influenza vaccine.

51. The method of claim 50, wherein the systemic solicited adverse events are below 50%.

52. The method of any one of claims 25-51, wherein the vaccine composition is administered to the human in a single-dose schedule.

53. The method of any one of claims 25-51, wherein the vaccine composition is administered to the human in a multiple-dose schedule.

54. The method of any one of claims 25-53, wherein the vaccine composition is administered to the human by intramuscular injection, subcutaneous delivery, intranasal delivery, oral delivery, intradermal delivery, transdermal delivery, transcutaneous delivery, or topical route.

55. A method of enhancing immunogenicity in a human at least 65 years of age, comprising administering to the human the vaccine composition of any one of claims 1-24, wherein the administration of the vaccine composition induces enhanced immunogenicity in the human compared to an administration of a standard-dose adjuvanted multivalent influenza vaccine.

56. The method of claim 55, wherein the enhanced immunogenicity is measured by one or more of: hemagglutination inhibition,
microneutralization, geometric mean titres, geometric mean ratios, and the percentage of subjects achieving seroconversion.

57. The method of any one of claims 55 and 56, wherein the enhanced immunogenicity appears by day 8 after vaccination/immunization/administering the vaccine composition.

58. The method of any one of claims 55-57, wherein the enhanced immunogenicity appears by day 8 and persists until at least day 22 after vaccination/immunization/administering the vaccine composition.

59. The method of any one of claim 55-58, wherein the enhanced immunogenicity appears by day 8 and persists until day 181 after vaccination/immunization/administering the vaccine composition.

60. The method of any one of claim 55-59, wherein the administration of the vaccine composition induces a higher seroconversion rate in the human compared to an administration of a standard-dose adjuvanted multivalent influenza vaccine.

61. The method of claim 60, wherein the higher seroconversion rate is 67.9%.

62. The method of any one of claims 55-61, wherein the administration of the vaccine composition does not increase the incidence of unsolicited adverse events compared to an administration of a standard-dose adjuvanted multivalent influenza vaccine.

63. The method of any one of claims 55-62, wherein preferably the administration of the vaccine composition does not increase the incidence of systemic solicited adverse events compared to an administration of a standard-dose adjuvanted multivalent influenza vaccine.

64. The method of claim 63, wherein the systemic solicited adverse events are below 50%.

65. A method for producing a vaccine composition, comprising admixing antigens from at least three or four different strains of influenza virus and an oil-in-water emulsion adjuvant, wherein the amount of the oil-in-water emulsion adjuvant is greater than an amount of an oil-in-water emulsion adjuvant in a standard-dose adjuvanted multivalent influenza vaccine.

66. The vaccine composition of any one of claims 1-24 for use in a method of inducing an immune response in a human at least 65 years of age, comprising administering the vaccine composition to the human at least 65 years of age.

67. The vaccine composition of any one of claims 1-24 for use in a method of enhancing immunogenicity in a human at least 65 years of age, comprising administering the vaccine composition to the human at least 65 years of age, wherein the administration of the vaccine composition induces enhanced immunogenicity in the human compared to an administration of a standard-dose adjuvanted multivalent influenza vaccine.

68. The vaccine composition of any one of claims 1-24 for use in treating or preventing influenza infection in a human at least 65 years of age.

69. The vaccine composition of any one of claims 1-24 for use in raising an immune response against influenza in a human at least 65 years of age.

70. The method or vaccine composition of any one of the preceding claims, wherein the vaccine composition increases immunogenicity against 1, 2, 3, or 4 of the antigens in the vaccine composition.
